(19) United States
(12) Patent Application Publication
(10) Pub. No.: US 2024/0066315 A1
(43) Pub. Date: Feb. 29, 2024

(54) Therapeutic devices and methods to improve body functions

(71) Applicant: Tianxin Wang, Walnut Creek, CA (US)

(72) Inventor: Tianxin Wang, Walnut Creek, CA (US)

(73) Assignee: Tianxin Wang, Walnut Creek, CA (US)

(21) Appl. No.: 18/386,602

(22) Filed: Nov. 3, 2023

Related U.S. Application Data
(63) Continuation of application No. 17/472,652, filed on Sep. 12, 2021.
Application No. 17/472,652 is a continuation-in-part of application No. 16/819,165, filed on Mar. 15, 2020, now abandoned.
(60) Provisional application No. 62/823,656, filed on Mar. 26, 2019.
(60) Provisional application No. 63/077,707, filed on Sep. 14, 2020.
(60) Provisional application No. 63/090,253, filed on Oct. 11, 2020.
(60) Provisional application No. 63/111,066, filed on Nov. 8, 2020.

(51) Int. Cl. A61N 5/06

(52) U.S. Cl. CPC A61N 5/0618; A61N 2005/0647; A61N 2005/0652; A61N 2005/0659; A61N 2005/0664

(57) ABSTRACT

Methods and therapeutic devices with noninvasive means for improving brain function including memory and sleep, improving eye health, improving digestive system health and improving immune functions are described. The non-invasive means is selected from electrical stimulation, heat stimulation, IR (infrared) radiation, mechanical stimulation or their combinations. In some embodiments, the method and device utilize stimulation to selected skin area on human body to achieve desired therapeutical effects.

### CROSS-REFERENCE TO RELATED APPLICATIONS

This application is a continuation application of U.S. application Ser. No. 17/472,652, filed on Sep. 12, 2021, which is a continuation-in-part application of U.S. application Ser. No. 16/819,165, filed on Mar. 15, 2020, which claims benefit of and priority to U.S. Provisional Patent Application Ser. No. 62/823,656, filed on Mar. 26, 2019 and is incorporated herein by reference in its entirety for all purposes. This application also claims benefit of and priority to U.S. Provisional Patent Application Ser. Nos. 63/077,707 filed on Sep. 14, 2020, 63/090,253 filed on Oct. 11, 2020, and 63/111,066 filed on Nov. 8, 2020, which are incorporated herein by reference in its entirety for all purposes. The entire disclosure of the prior applications are considered to be part of the disclosure of the instant application and are hereby incorporated by reference.

### BACKGROUND OF THE INVENTIONS

Dementia is a broad category of brain diseases that cause a long-term and often gradual decrease in the ability to think and remember that is severe enough to affect daily functioning. Other common symptoms include emotional problems, difficulties with language, and a decrease in motivation. A dementia diagnosis requires a change from a person's usual mental functioning and a greater decline than one would expect due to aging. The most common type of dementia is Alzheimer's disease, which makes up 50% to 70% of cases. Other common types include vascular dementia, dementia with Lewy bodies, frontotemporal dementia, normal pressure hydrocephalus, Parkinson's disease dementia, and Creutzfeldt-Jakob disease. Currently there is no known cure for dementia. US patent application US20190126062A1 disclosed devices, systems, and methods for treating dementia or Alzheimer's disease in a subject in need using visual/audio stimuli having a frequency of about 30 Hz to about 50 Hz, and more specifically about 40 Hz, are non-invasively delivered to the subject to entrain gamma oscillations in multiple brain regions of the subject, including the prefrontal cortex (PFC) and the hippocampus.

### SUMMARY OF THE INVENTIONS

This application discloses methods and therapeutic devices with non-invasive means for improving brain function such as improving sleep and memory, and treating senile dementia including Alzheimer's disease. The non-invasive means is selected from light stimulation, electrical stimulation, heat stimulation, IR (infrared) radiation, mechanical stimulation (e.g. sound/vibrational stimulation) or their combinations. In some embodiments, the invention utilizes electrical current passed through skin including acupuncture sites on human head or hand to achieve the desired therapeutical and beneficial effects. It also provides methods and devices to improve sleep quality including inducing deep sleep mode such as slow wave sleep, improving eye health, improving digestive system health and improving immune functions.

In some embodiments the device and method in the current invention is directed towards stimulating one or more of areas including acupuncture points selected from wrist, waist, arm, neck, head, face, forehead, hand, feet, finger, toe, palm, sole, Taiyang, Yangbai, Yintang, Baihui (DU/GV20), Sishencong (EX-HN1), Fengchi (GB 20), TaiYang (EX-HN5) and Shenting (DU/GV 24) for the purpose of improving brain function and treating senile dementia including Alzheimer's disease. Preferably the device in the invention is portable and self-contained, with means to be attached to the head or other body part. It can be in the form of hat or hairband or glass or helmet form. The device can produce electrical pulse or heat or light or IR radiation or vibration or sound or their combination and apply them to the said skin area such as acupuncture points.

In some embodiments, the light and/or electrical stimulation is also applied to the inside surface area of ear or nose or eye or eye lid. A mean that can deliver these stimulation (e.g. electrode, light source having ear cover/insert/plug or nasal cover/insert or eye mask, eye glass shape/format) is also incorporated within the said device.

In one aspect, the methods and devices described in the current invention use electro pulse or electrical pulse in combination with vibration/sound or heat or light such as IR radiation applied to said body areas and/or said acupuncture points or other area in the head to achieve the desired therapeutical effects. A person desiring to improve brain function (e.g. reduce aging, improving memory and sleep, improving blood flow, improving metabolization and detoxification in brain, reducing brain function impairment, reducing cognitive impairment, improving learning, improving reasoning and problem solving, improving sensory processing, improving brain tissue growth, improving neuron/nerve integrity, growth, function, activity and function or treating dementia such as Alzheimer's disease or vascular dementia) places the electrodes on one or more of areas and the acupuncture points selected from wrist, waist, arm, hand, palm, neck, head, face, forehead, hand, feet, finger, toe, palm, sole, acupuncture points including Taiyang, Yangbai, Yintang, Baihui, Sishencong, Fengchi, and Shenting and applies electrical stimulation through these electrodes. Heat and/or light and/or IR and/or vibration and/or sound stimulation produced by the device can also be applied to these areas and acupuncture points for these purpose to improve brain function and/or reduce symptoms of dementia. The same device and method also be used to treat depression and insomnia, as well as to improve sleep including inducing deep such as slow wave sleep, which will also improve memory consolidation and improve cerebrospinal fluid flow that can clean metabolic products and toxic wastes (e.g. amyloid) from the brain for better brain health. The stimulation pulse generating circuitry and power supply can be most conveniently packaged in a housing structure with means to affix to the head or other body part such as a hat or helmet shape structure. One or more electrodes are on the inner surface of the housing which is placed in contact with skin. The device is placed so that the electrode/electrodes overlie the target skin area or acupuncture points known to affect a desired therapy. Alternatively, the device is placed so that the electrodes is close or overlie a nerve that runs under the acupuncture point, in which case the device may be placed some distance from the associated acupuncture point to provide a comfortable placement for the device. It can also be attached to other areas on the head or other body area.

The device can also contain one or more built in heating element that can keep the temperature of the skin contact surface between 40˜60° C. to provide a heating effect to the stimulation sites/area. The device can also contain one or more built in visible light and/or IR radiation element that can produce a light and pass it to the skin contact surface to provide a visible light and/or IR stimulation effect to the stimulation sites/area as well as means to provide mechanical (e.g. sound) stimulation.

### BRIEF DESCRIPTION OF THE DRAWINGS

FIG. 1A shows an example of stimulation frequency in cycled pattern.

FIG. 1B shows examples amplitude modulated wave and frequency modulated wave.

FIG. 1C show an example pulse width modulations.

FIG. 2A shows an example of block diagram of the electrical circuitry suitable for the devices without additional heating elements.

FIG. 2B show an example of block diagram of the electrical circuitry suitable for the devices with heating elements.

FIG. 3 illustrates an example of block diagram of the electrical circuitry suitable for the devices with electrodes, heating elements, speaker and vibrator.

FIG. 4 shows an example of the device electrode assembly.

FIG. 5 shows examples of another type of device electrode assembly.

FIG. 6 shows examples of device electrode assembly with heating element.

FIG. 7 shows examples of device electrode assembly with light radiation element.

FIG. 8 shows examples of electrode assembly with IR radiation and heating function.

FIG. 9 is an illustration of an example of the device of the invention as worn on the head of a person using the device.

FIG. 10 shows examples of stimulation points on the head.

FIG. 11 shows an example of device containing an array of electrode, LED and heating element with additional speaker.

FIG. 12 shows formats of device with a shape configured to be wrapped around neck

FIG. 13 shows examples of device configurations that can be used

FIG. 14 shows exemplary formats of the device that can be attached to head

FIG. 15 shows additional formats of the device having helmet like configuration

FIG. 16 shows another helmet like device configurations

FIG. 17 shows device configurations with stimulation output end mounted on sliding rail

FIG. 18 shows exemplary format of a device to improve eye health

FIG. 19 shows the skin contact side of a device to improve eye health

FIG. 20 shows exemplary format of the device in the form of wearable garment with multiple LED lights

FIG. 21 shows exemplary format of a vest like wearable device with multiple LED lights and additional speakers or vibrators

FIG. 22 shows exemplary format of a vest like wearable device with additional extension at the bottom and a short like wearable device

FIG. 23 shows exemplary devices to stimulate lymph node area

FIG. 24 shows examples of insertable device

FIG. 25 shows an insertable device to treat hemorrhoid

FIG. 26 shows chair seat cover like devices to treat hemorrhoid

### DETAILED DESCRIPTION

In some embodiments, the preferred (but not limited to) electrical pulse frequency used by the method and device of the current invention is between 2-100 Hz or the combinations of the frequency within 2-100 Hz; the preferred pulse width is 0.001-50 ms and the preferred current is 0.5-100 milliamperes. In some embodiments, exemplary preferred electrical pulse repetition rate of approximately 2 pulses per second with a pulse width of 600 microseconds, or approximately 15 pulses per second with a pulse width of 300 microseconds, or approximately 40 pulses (e.g. 30-50 or 35˜45 pulses) per second with a pulse width of 200 microseconds, or approximately 100 pulses per second with a pulse width of 200 microseconds, or the combination of the above wave patterns can provide effective result. The preferred electrical power levels can be about 0.5-50 milli-amps peak pulse height or other power level that the subject in need feel comfortable. A wider range of pulse patterns can also be used in these non-invasive stimulation devices. Bi-directional electric pulse is preferred to avoid skin damage. In general, 10-60 min stimulation daily or on demand can provide effective treatment to the person in need.

In other examples, medium-frequency currents are used. Typical medium frequency (e.g. 1k-10 kHz) can stimulate the muscle and provide a massage like feeling to the area treated. In order to interrupt the current after each depolarization, rhythmically increasing and decreasing the amplitude (amplitude modulation) can be applied. The amplitude modulation frequency (AMF) determines the frequency of the depolarization. The AMF corresponds to the frequencies used in low-frequency electrotherapy. The amplitude modulation can also be replaced by pulse width modulation.

The electric current pattern used in modulated medium frequency electrotherapy can also be used in the devices of the current invention. Suitable frequency can be 2-10 kHz. The combination of low frequency and medium frequency can be used, e.g. the combination of frequency of 1-50 Hz low frequency current and 2k-10 kHz medium frequency current. The low and/or medium frequency can have varieties of wave shape/pattern (e.g. sinusoidal wave, squire wave, continued, discontinued, varying amplitude, symmetrical wave or unsymmetrical wave etc.). The amplitude of medium frequency can vary at the low frequency pattern. In some embodiments, suitable current is between 0.05-5 mA.

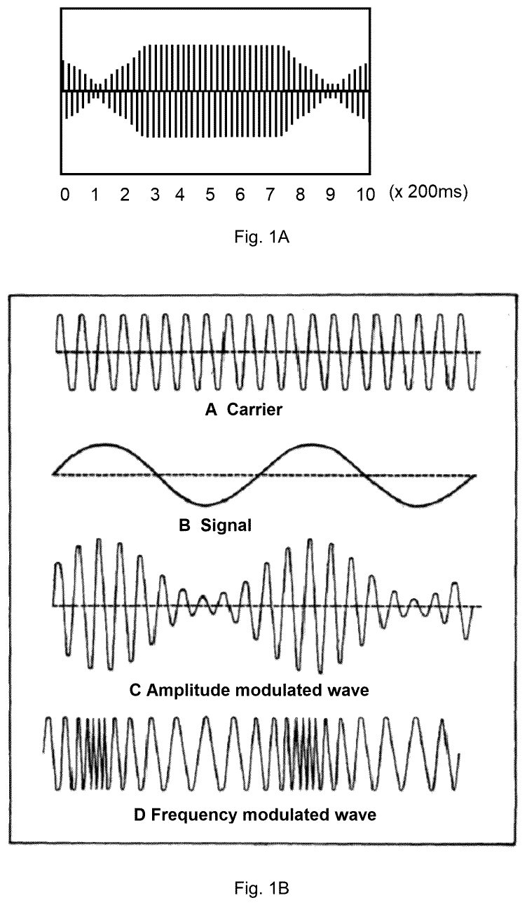

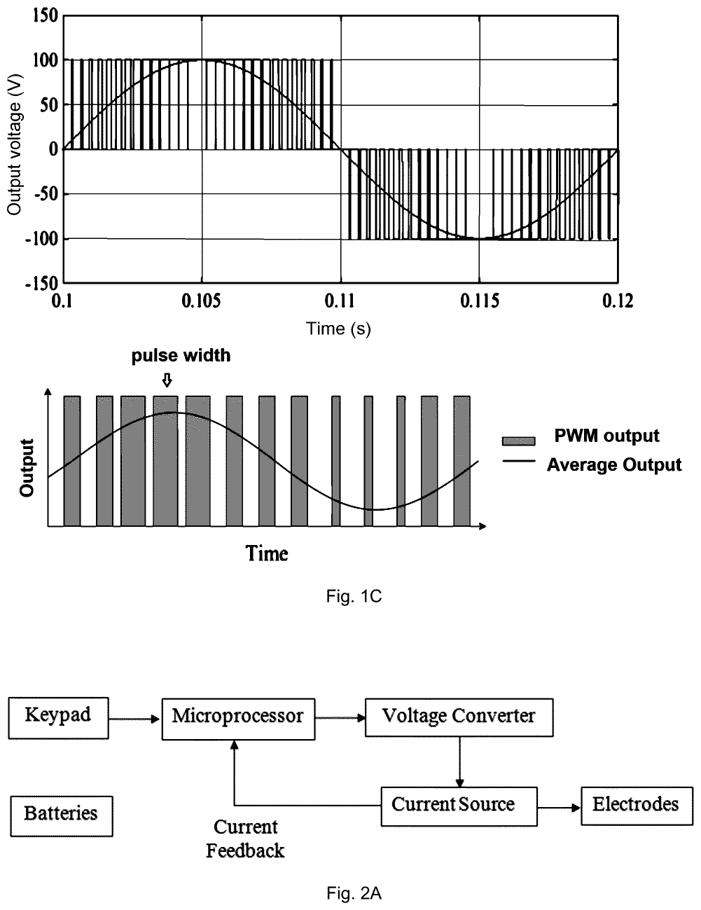

A gamma wave is a pattern of neural oscillation in humans with a frequency between 25 and 100 Hz, though 40 Hz is typical. It can benefit and improve brain function. Light, sound, vibration, electric frequency, electromagnetic or magnetic stimulation that can stimulate the gamma wave in brain can be used for the current invention, for example between 25-100 Hz, preferably between 30-80 Hz. Stimulation having a frequency can induce brain to resonance in the similar frequency. In some embodiments, the stimulation frequency such as electrical or light or sound pulse or other mechanical (e.g. vibrational) frequency is between 30-60 Hz. In some embodiments, the stimulation frequency such as electrical pulse frequency is between 35-50 Hz. In some embodiments, the stimulation frequency such as electrical pulse frequency is 40 Hz. In some embodiments, the frequency is 40 Hz and the peak width is between 0.1-0.6 ms, e.g. 0.3 ms for electrical pulse. Peak width for sound, light and other mechanical stimulation can be different, e.g. 1 ms-10 ms. In some embodiments, the stimulation frequency such as electrical pulse is applied in cycled batch, in each cycled batch the current start from low to high and then keep constant for certain time such as 1-10 s, then drop to low gradually for the next cycled batch. For example as shown in FIG. 1A, a ˜40 Hz stimulation pulse starts from low to high in ˜300 ms and then keep constant for ˜800 ms, then drop to low gradually in ˜300 ms until the next cycled batch. There could be an interval period (no stimulation pulse) between each cycled batch, e.g. 1-10 seconds. The combination of different frequency of gamma wave can also be used, for example the frequency is 35 Hz in the first 5 min and then 40 Hz for 5 min and then 45 Hz for 5 min and then goes back to 40 Hz for 10 min.

In some embodiments, medium-frequency stimulation such as electrical current or light or sound or vibration are used to give gamma wave stimulation. Typical medium frequency (e.g. 1k-10 kHz as carrier wave frequency) can be used. In order to give gamma wave stimulation, rhythmically increasing and decreasing the amplitude (amplitude modulation) can be applied at gamma wave frequency. The amplitude modulation frequency (AMF) determines the frequency of the gamma wave signal. The AMF corresponds to the frequencies used in low-frequency signal stimulation used in therapy. Therefore, the stimulation such as electric current pattern used in modulated medium frequency electrotherapy can also be used in the devices of the current invention. Suitable carrier frequency can be 2k-10 kHz. The amplitude of medium frequency can vary at the low frequency pattern at the frequency of gamma wave to generate gamma wave stimulation. The combination of low frequency and medium frequency can be used, e.g. the combination of low frequency of 30-100 Hz gamma wave frequency stimulation such as electric current stimulation and 2k-10 kHz medium frequency such as electric current stimulation. The low and/or medium frequency can have varieties of wave shape/pattern (e.g. sinusoidal wave, squire wave, continued, discontinued, varying amplitude, symmetrical wave or unsymmetrical wave etc.). In some examples, suitable current is between 0.05-5 mA. If other stimulation is used such as light or sound, the intensity used should be tolerable by the user or be comfortable to the user. FIG. 1B shows exemplary pattern of amplitude modulated wave and frequency modulated wave.

Similarly, a light stimulation can also be given to a subject in need for the same indication. The light stimulation can use medium or high frequency light to give gamma wave stimulation. Typical medium frequency (e.g. 1k-10 kHz or higher as carrier frequency) can be used. In order to give gamma wave stimulation, rhythmically increasing and decreasing the amplitude of light intensity (amplitude modulation) can be applied at gamma wave frequency. The amplitude modulation frequency (AMF) determines the frequency of gamma wave. The AMF corresponds to the frequencies used in low-frequency light stimulation. Therefore, the light pattern in modulated medium frequency flash (flicker) can be used in the devices of the current invention. Suitable carrier frequency can be 2k-10 kHz or higher frequency such as 10k-1 MHz. In some embodiments the carrier frequency is between 2k-10 kHz. The amplitude of medium frequency can vary at the low frequency pattern at the frequency of gamma wave to generate gamma wave stimulation. The combination of low frequency and medium frequency can be used, e.g. the combination of low frequency of 30-100 Hz (such as 40 Hz) gamma wave frequency light flicker and 2k-10 kHz medium frequency light flicker. The low and/or medium frequency can have varieties of wave shape/pattern (e.g. sinusoidal wave, squire wave, continued, discontinued, varying amplitude, symmetrical wave or unsymmetrical wave etc.). The amplitude of medium frequency can vary at the low frequency pattern. The simulation intensity and duration of stimulation used should be tolerable by the user or be comfortable to the user.

Similarly, a sound and/or vibration (sound/vibration) or other mechanical stimulation can also be given to a subject in need for the same indications. The sound/vibration or other mechanical stimulation can use medium or high frequency sound/vibration to give gamma wave stimulation. Typical medium frequency (e.g. 1k-20 kHz) can be used. In order to give gamma wave stimulation, rhythmically increasing and decreasing the amplitude of sound/vibration or other mechanical stimulation intensity (amplitude modulation) can be applied at gamma wave frequency. The amplitude modulation frequency (AMF) determines the frequency of gamma wave. The AMF corresponds to the frequencies used in low-frequency sound/vibration stimulation. Therefore, the sound/vibration or other mechanical stimulation pattern in modulated medium frequency can be used in the devices of the current invention. Suitable frequency can be 2k-10 kHz or higher frequency such as 10k-100 kHz. Lower frequency such as 100 Hz-1 kHz can also be used. In some embodiments, Suitable carrier frequency can be 0.2k-10 kHz. The amplitude of medium frequency can vary at the low frequency pattern at the frequency of gamma wave to generate gamma wave stimulation. The combination of low frequency and medium frequency can be used, e.g. the combination of low frequency of 30-100 Hz (such as 40 Hz) gamma wave frequency sound/vibration/other mechanical stimulation and 2k-10 kHz medium frequency sound/vibration/other mechanical stimulation. The low and/or medium frequency can have varieties of wave shape/pattern (e.g. sinusoidal wave, squire wave, continued, discontinued, varying amplitude, symmetrical wave or unsymmetrical wave etc.). The amplitude of medium frequency can vary at the low frequency pattern. The intensity and duration of stimulation used should be tolerable by the user or be comfortable to the user.

Alternatively, frequency modulated wave (FMF) or PWM (pulse width modulation) stimulation can also be used instead of or in combination with amplitude modulation frequency (AMF) to achieve the desired low frequency stimulation (e.g. ˜40 Hz or delta wave frequency) using medium carrier frequency (e.g. 1k-10 kHz) or high frequency stimulation for light or electric or sound/vibration/other mechanical stimulation or visible light or IR stimulation. PWM (pulse width modulation) is widely used in OLED display. The similar technique can be readily adopted for the current invention. FIG. 1C shows several format of pulse width modulations. By varying the pulse width at the switching frequency in PWM, stimulation at desired frequency (e.g. ˜40 Hz gamma frequency or delta wave frequency) can be achieved. For example, suitable PWM switching frequency can be 1k-10 kHz or higher frequency such as 10k-1M kHz. In some embodiments, Suitable PWM switching frequency can be 10k-100 kHz. The low and/or high frequency can have varieties of wave shape/pattern (e.g. sinusoidal wave, squire wave, continued, discontinued, varying amplitude, symmetrical wave or unsymmetrical wave etc.). The amplitude of PWM frequency can vary at the low frequency pattern. The intensity and duration of stimulation used should be tolerable by the user or be comfortable to the user.

In some embodiments, the device and method of the current invention use alpha (7.5-14 Hz) or theta (4-7.5 Hz) or delta (around 0.1-4 Hz) wave frequency/pulse stimulation or their combination instead of using gamma wave stimulation. The stimulation can be selected from electric pulse, electromagnetic wave, magnetic field, visible light, IR, sound/vibration/other mechanical stimulation or their combinations. Stimulation having a frequency can induce brain to resonance at similar frequency. To induce brain wave at alpha (7.5-14 Hz) or theta (4-7.5 Hz) or delta (0.5-4 Hz) or zeta (0.1-0.5 Hz), the stimulation can have the frequency at alpha (7.5-14 Hz) or theta (4-7.5 Hz) or delta (0.5-4 Hz) or zeta (0.1-0.5 Hz) respectively. The format and generation of these alpha (7.5-14 Hz) or theta (4-7.5 Hz) or delta (0.5-4 Hz) or zeta (0.1-0.5 Hz) stimulation can be similar to those in gamma wave stimulation described previously, e.g. can be directly at the frequency of alpha (7.5-14 Hz) or theta (4-7.5 Hz) or delta (0.5-4 Hz) or zeta (0.1-0.5 Hz) wave or indirectly using amplitude modulation frequency (AMF) or FMW (frequency modulated wave) or PWM (pulse width modulation) frequency or their combination similar to those in gamma frequency described previously. Although delta wave frequency is described as 0.5-4 Hz frequency in some literatures, zeta (0.1-0.5 Hz) wave frequency is considered as a subset of delta wave frequency. The difference of frequency between different brain wave is not distinct and there is no clear definition. For example, some publications define delta wave frequency as those <3 Hz instead of 0.5-4 Hz or 0.1-4 Hz. In the current inventions, delta wave frequency is defined as a wave having frequency between 0.1-4 Hz.

In embodiments of the current inventions, the stimulation frequency such as electrical or light or sound/vibration pulse frequency or other mechanical stimulation (for example, applying mechanical force/pressure periodically at certain frequency is considered vibration) is between 0.1-4 Hz to induce delta wave. The term mechanical stimulation in the current inventions include sound/vibration stimulation. Sound stimulation is essentially a vibration stimulation. The term vibration in the current inventions include mechanical stimulation such as applying mechanical force/pressure to target area periodically at certain frequency (e.g. pulse). The term vibrator means a physical mean that can generate a vibration to a target area. Example of vibrators can be found in the device to provide massaging effect such as those used in commercial massagers. For example, the vibrator can be a device that provides kneading effect such as shiatsu. Rotating wheel or inflatable airbag can be used to apply pressure to body part to provide massage effect. The inflatable airbag can inflate and deflate periodically to provide cycled pressure as massaging effect. In some embodiments, the delta wave stimulation frequency such as electric pulse frequency, light pulse frequency, vibration frequency is between 1-3 Hz. In some embodiments, the stimulation frequency such as electric pulse frequency, light pulse frequency, vibration frequency is 2 Hz. In some embodiments, the stimulation frequency such as electrical pulse frequency, light pulse frequency, vibration frequency varies between 0.5-4 Hz repeatedly. In some embodiments, the electrical pulse frequency is 0.3-2 Hz and the peak width is between 0.1-10 ms, e.g. 1 ms. In some embodiments, the light or vibration pulse frequency is 1-2 Hz and the peak width is between 10-400 ms, e.g. 200 ms. In some embodiments, the light or vibration pulse frequency is 0.5-1 Hz and the peak width is between 100-1000 ms, e.g. 500 ms. In some embodiments, the stimulation such as electrical pulse, light pulse, vibration is applied in cycled batch, in each cycled batch the current start from low to high and then keep constant for certain time such as 5-10 s, then drop to low gradually for the next cycled batch as shown in the FIG. 1A. There could be an interval period (no electric or light or vibration pulse) between each cycled batch, e.g. 1-10 seconds. The combination of different frequency of delta wave can also be used, for example the frequency is 3 Hz in the first 15 min and then 2 Hz for 15 min and then 1 Hz for 15 min and then and then 0.5 Hz for 15 min goes back to 1 Hz for 15 min. The combination of different brain wave stimulation can also be used. In some embodiments the stimulation is applied at the order of reduced frequency, for example optional gamma (35˜45 Hz) wave stimulation for 15 min and then alpha (7.5-14 Hz) wave stimulation for 15 min and then theta (4-7.5 Hz) wave stimulation for 10 min and then delta wave stimulation for 30 min.

In some embodiments, medium-frequency stimulation such as electrical current or light or sound or vibration are used to generate delta wave stimulation. Typical medium frequency (e.g. 1k-10 kHz as carrier wave frequency) is used. In order to give delta wave stimulation, rhythmically increasing and decreasing the amplitude (amplitude modulation) can be applied at delta wave frequency. The amplitude modulation frequency (AMF) determines the frequency of the delta wave signal. The AMF corresponds to the frequencies used in low-frequency signal stimulation used in therapy. Therefore, the stimulation such as electric current pattern used in modulated medium frequency electrotherapy can also be used in the devices of the current invention. Suitable carrier frequency can be 1k-50 kHz. The amplitude of medium frequency can vary at the low frequency pattern at the frequency of delta wave to generate delta wave stimulation. The combination of low frequency and medium frequency can be used, e.g. the combination of low frequency of 0.1-4 Hz delta wave frequency stimulation such as electric current and 2k-10 kHz medium frequency such as electric current. The low and/or medium frequency can have varieties of wave shape/pattern (e.g. sinusoidal wave, squire wave, continued, discontinued, varying amplitude, symmetrical wave or unsymmetrical wave etc.). In some examples, suitable current is between 0.05-5 mA. If other stimulation is used such as light or sound, the intensity used should be tolerable by the user or be comfortable to the user.

Similarly, a delta wave light stimulation can also be given to a subject in need for the same indications. The light stimulation uses medium or high frequency light to give delta wave stimulation. Typical medium frequency (e.g. 1k-10 kHz or higher as carrier frequency) can be used. In order to give delta wave stimulation, rhythmically increasing and decreasing the amplitude of light intensity (amplitude modulation) can be applied at delta wave frequency. The amplitude modulation frequency (AMF) determines the frequency of delta wave. The AMF corresponds to the frequencies used in low-frequency light stimulation. Therefore, the light pattern in modulated medium frequency flash (flicker) can be used in the devices of the current invention. Suitable carrier frequency can be 2k-10 kHz or higher frequency such as 10k-1 MHz. In some embodiments suitable carrier frequency can be 2k-10 kHz. The amplitude of medium frequency can vary at the low frequency pattern at the frequency of delta wave to generate delta wave stimulation. The low and/or medium frequency can have varieties of wave shape/pattern (e.g. sinusoidal wave, squire wave, continued, discontinued, varying amplitude, symmetrical wave or unsymmetrical wave etc.). The amplitude of medium frequency can vary at the low frequency pattern. The simulation intensity and duration of stimulation used should be tolerable by the user or be comfortable to the user.

Similarly, a sound and/or vibration (sound/vibration) stimulation can also be given to a subject in need for the same indications. The sound/vibration stimulation can use low or medium or high frequency sound/vibration to give delta wave stimulation. Typical low frequency (e.g. 20 Hz-1K Hz) or medium frequency (e.g. 1k-20 kHz) can be used. In order to give delta wave stimulation, rhythmically increasing and decreasing the amplitude of sound/vibration intensity (amplitude modulation) can be applied at delta wave frequency. The amplitude modulation frequency (AMF) determines the frequency of delta wave. The AMF corresponds to the target frequencies used in sound/vibration stimulation. Therefore, the sound/vibration pattern used in modulated low/medium frequency can be used in the devices of the current invention. Suitable carrier frequency can be 2k-10 kHz or higher frequency such as 10 k-100 kHz. Lower carrier frequency such as 20 Hz-1 kHz can also be used. In some embodiments, Suitable carrier frequency can be 20 Hz-10 kHz. In some embodiments, Suitable carrier frequency can be 100 Hz-1 kHz. The amplitude of carrier frequency can vary at the low frequency pattern at the frequency of delta wave to generate delta wave stimulation. The combination of low frequency and medium frequency can be used, e.g. the combination of low frequency of 0.1-5 Hz (such as 2 Hz) delta wave frequency sound/vibration and 2k-10 kHz medium frequency sound/vibration. The low and/or medium frequency can have varieties of wave shape/pattern (e.g. sinusoidal wave, squire wave, continued, discontinued, varying amplitude, symmetrical wave or unsymmetrical wave etc.). The amplitude of medium frequency can vary at the low frequency pattern. The intensity and duration of stimulation used should be tolerable by the user or be comfortable to the user.

Alternatively, PWM (pulse width modulation) or FMW stimulation can also be used instead of or in combination with amplitude modulation frequency (AMF) to achieve the desired low frequency stimulation (e.g. ˜2 Hz) using low/medium frequency (e.g. 1 Hz-10 kHz) or high frequency stimulation for light or electric or sound/vibration or visible light or IR stimulation.

PWM (pulse width modulation) is widely used in OLED display. The similar technique can be readily adopted for the current invention. By varying the pulse width at the carrier frequency (e.g. ˜2k Hz frequency), stimulation at desired frequency (e.g. ˜2 Hz delta frequency) can be achieved. For example, suitable PWM switching frequency can be 1k-10 kHz or higher frequency such as 10k-1M kHz. In some embodiments, Suitable PWM switching frequency can be 10k-100 kHz. The low and/or high frequency can have varieties of wave shape/pattern (e.g. sinusoidal wave, squire wave, continued, discontinued, varying amplitude, symmetrical wave or unsymmetrical wave et ac.). The amplitude of PWM frequency can vary at the low frequency pattern. The intensity and duration of stimulation used should be tolerable by the user or be comfortable to the user.

Humans can detect audio sounds in a frequency range from about 20 Hz to 20 kHz. To generate gamma wave sound stimulation, an audio sound having a vibration frequency at gamma wave frequency (e.g. 35-50 Hz) can be used directly since it is within the frequency range human ear can hear. Alternatively, sound having one or more carrier wave frequency (e.g. 100 Hz to 10 Kz) other than gamma wave frequency can be used, and the gamma wave frequency stimulation is provided by modulating the carrier wave using amplitude modulation wave or pulse width modulation or frequency modulated wave at gamma frequency (e.g. 35-50 Hz). The carrier sound can be in the form of single tone/syllable or the combination of multiple tone/syllable or melody.

Although mechanical stimulation at delta wave frequency such as vibration stimulation at 0.1-4 Hz to skin is feasible, sound at delta wave frequency (0.1-4 Hz) is not within the range that human can hear. To generate delta wave audio sound stimulation that human can hear, sounds having one or more carrier wave frequency (e.g. 20 Hz to 20 Kz) that within human hearing range can be used, and the delta wave frequency stimulation is provided by modulating the carrier sound wave using amplitude modulation wave or pulse width modulation or frequency modulated wave at delta frequency (e.g. 0.1-4 Hz). The carrier sound can be in the form of single tone/syllable or the combination of multiple tone/syllable or melody.

Current invention disclose device having means to provide electric pulse or electromagnetic wave or magnetic field or visible light or IR or sound/vibration or their combinations to improve brain function and body functions. The device contains means to deliver said stimulation at said brain wave frequency. In some embodiments, the device contains means to produce electric pulse. The device can also contain one or more built in heating means that can keep the temperature of the skin contact surface between 35˜60 degree to provide a heating effect to the stimulation sites/area. The heating means can be an electrical heating element powered by a battery outside power supply source. The preferred heating temperature at the surface is between 40˜55 degree. In some embodiments the heating temperature at the surface is between 40˜50 degree. A preferred heating element is self-regulating heater such as polymer PTC heating elements or ceramic PTC heater or carbon fiber/sheet heater. Resistive heaters can be made of conducting PTC rubber materials or ceramic PTC material where the resistivity increases exponentially with increasing temperature. Such a heater will produce high power when it is cold, and rapidly heat up itself to a constant temperature. Due to the exponentially increasing resistivity, the heater can never heat itself to warmer than this set temperature. The temperature can be chosen during the production of the rubber or ceramic. The heating step can be cycled to avoid skin desensitization. For example, each heating step is 1-10 min followed by 0.5-3 min none heating period.

The device can also contain one or more built in visible light and/or IR radiation element that can produce visible light and/or IR radiation for either thermal or non-thermal effect and pass it to the skin contact surface or area of interest (e.g. to provide a photon stimulation and/or heating effect to the stimulation sites/area. The visible light or IR radiation elements can be tungsten wire, carbon, or alloys of iron, chromium, and aluminum as well as LED and laser or their combinations to provide broader wavelength coverage. IR radiation elements are widely used in physical therapy and they can be adapted readily for the current invention. The device can have one centralized visible light and/or IR radiation elements connected to multiple optical fibers, which can transfer the visible light and/or IR radiation to the desired stimulation sites. The surface of the housing can have one or more light transparent windows to allow the photon emitted from the light source reaching the skin. Suitable wavelength of IR can be between 700 nmm-1 mm. It can be either NIR (near IR) or MIR or FIR or its combination. In some embodiments, it is between 800 nm-100 um. In other examples, it is between 1 um-20 um. The output power of IR radiation can be adjusted to provide effective and safe radiation. When heating effect is desired, preferably the suitable power level can heat the target skin site to 40° C.-50° C. but does not burn/damage the skin and is acceptable by the user. The radiation can be either continuous or pulsed as those used in the electric pulse stimulation described previously. The intensity and duration of stimulation used should be tolerable by the user or be comfortable to the user.

In one example, a 10 W IR radiation element with 2 μm-10 μm wavelength output is used; the output is coupled to IR optical fibers to deliver IR radiation to the treatment sites. In another example, a 808 nm GaAlAs laser or 1550 nm laser can be used, connected to multiple optical fiber to deliver IR pulse to the sites and the gamma wave frequency between 30-50 Hz at the power level of 10-1000 mW/cm² can be applied. When IR LED is used, each stimulation site can have its own IR LED attached without the need of using optical fibers. Red light and NIR radiation can penetrate into deep tissue and improve mitochondrial activity and activate cell energy metabolism.

In some embodiments, the current invention discloses a method for improving brain function and other body functions in a subject in need thereof, the method comprising administering a non-invasive electrical stimulus have a frequency of about 35 Hz to about 45 Hz to the subject's surface area on the head as well as a frequency of about 0.1 Hz to about 4 Hz such as 0.5-4 Hz. Additional heat and/or NIR and sound stimulation can also be applied to the head. The current invention also discloses a device that have means to generate a non-invasive electrical stimulus having a frequency of about 35 Hz to about 45 Hz as well as a frequency of about 0.1 Hz to about 4 Hz such as 0.5-4 Hz to be applied to the subject's surface area on the head for improving brain function. The device contains an electrical stimulus generating source and a fixture to be attached to the head.

The current invention also discloses a method for improving brain function in a subject in need thereof, the method comprising administering a non-invasive light stimulus have a frequency of about 0.1 Hz to about 5 Hz such as 0.5-4 Hz to the subject's surface area on the head. Additional heat stimulation can also be applied to the head. The light can be white light, red light or near IR light. The current invention also discloses a device that have means to generate a non-invasive light stimulus having a frequency of about 0.1 Hz to about 5 Hz such as 0.5-4 Hz to be applied to the subject's surface area on the head for improving brain function. The device contains a light stimulus generating source and a fixture to be attached to the head.

The method for improving brain function can also be combination of electrical stimulus, heat, sound/vibration and visible light/IR (non-heating effect) described previously. In general, higher and longer stimulation level can provide better effect but the intensity and duration of stimulation used should be tolerable by the user or be comfortable to the user and should not cause damage to the treated area/tissue and the subject. The user can adjust the device output intensity to achieve the desired level. Different type of stimulation can be applied simultaneously or sequentially. For example, electric stimulation and light stimulation can be applied to a subject at the same time concurrently, or they can be applied to a subject alternately or sequentially.

To improve brain function and cell activity, the wavelength of the light to irradiate head area (none-eye area) can be between 500-1500 nm, or 550-1100 nm such as those having peak wavelength at 590 nm or 670 nm or 780 nm or 790 nm or 808 nm or 810 nm or 850 nm or 980 nm or 1064 nm or their combinations. In some embodiments it is selected from red light/NIR (near IR) such as those with peak at 633, 670, 810, 850, 980 and 1064 nm. In some embodiments it has a peak wavelength at 808-820 nm. In some embodiments it has a peak wavelength at 633 nm or nm or 810 nm or 850 nm. In some embodiments the light intensity can be 10-1000 mW/cm². For example, they can be multiple light emitting units such as LEDs having 0.5-6 W power level each with total power of 5-200 W, and the light output of each light emitting unit (e.g. LED) can be 50-600 lumens each. In some embodiments the irradiation can be 1 min-100 min 1-3 times a day for 1-10 weeks or until desired effect is achieved. The intensity of the light needs to be not too high that can cause damage to the skin of the irradiation area. The light stimulation can be in the frequency pattern described previously or non-pulsed (continuous).

When light irradiation to open eye or eye lid is applied to improve brain function, the intensity needs to be lower to the extent that the user feels comfortable and does not cause damage to the eye (e.g. 5-100 mW/cm²). When irradiation to open eye or eye lid or nose or ear duct is applied to improve brain function, the wavelength is not limited to red light/NIR, it can be any visible light and/or IR light. The light stimulation can be in the frequency pattern described previously.

The current invention also disclosed a method and device to improve vision, improve eye health, reduce fatigue, treating dry eye, reduce eye inflammation, AMD and improve tissue and wounds repairing of the eye by providing visible light or IR light irradiation to eye lid (can be closed eyelid) and/or eye area as well as heating simulation to eye area using a device that has a mean to mount on the eye area. The light stimulation and the heat stimulation can be applied simultaneously or sequentially. Electrical stimulation can also be applied to the skin in the eye area or nearby area such as related acupuncture sites (e.g. Taiyang, Jingming, Sibai and Sizukong). The device contains a light generating means and a heating means such as those described previously for head stimulation as well as a mounting means to mount it to the eye area. Electrical current generating means to provide pulse electric stimulation such as those described previously can also be incorporated.

The wavelength of the light can be between 550-1100 nm such as those having peak wavelength at 590 nm or 670 nm or 780 nm or 790 nm or 810 nm or 850 nm or 980 nm or 1064 nm or their combinations. The light irradiation can be directed to opened eye or closed eye through eye lid. The wavelength of the light can be between 550-1100 nm such as the wavelength selected from nm, 633 nm, 660 nm, 670 nm, 689 nm, 780 nm, 790 nm, 810 nm, 850 nm, 980 nm, 1064 nm or their combinations. It can also be continuous wave white light or yellow light if it is applied to closed eye lid. When white light or yellow light pass through eye lid, it become red light abundant in wavelength as other wavelength of light are absorbed by eye lid. When open eye light irradiation is applied, in some embodiments the intensity is between 1-100 mW/cm² at the cornea with a total dose of 0.1 J˜50 J/cm² each treatment, the irradiation can be 5-500 seconds 1-3 times a day for 1-10 weeks or until desired effect is achieved. In some embodiments the intensity is between 20-50 mW/cm². In some embodiments the intensity is between 40 mW/cm². When closed eye irradiation is applied, high light intensity can be applied to the extent that the user feels comfortable and the irradiation time can also be longer to the extent that the user feels comfortable such as a total density is between 5-50 J/cm² at eye lid.

The device of the current invention can have a communication module that can communicate with an external control (command) module to receive commands of the stimulation output (e.g. stimulation type, time, frequency, on/off, power level and pulse pattern) and produce stimulation accordingly. The external control module can be a remote or computer or a cell phone with dedicated application installed. The communication can utilize Wi-Fi or blue tooth or IR or radio signal. The device can have an on/off control to turn on or turn off the communication module.

The electrical circuitry of the device can be implemented with well-known art. There are many designs to implement the circuitry. FIG. 2A illustrates an example of the circuit diagram suitable for the device. The microprocessor receives input from the keypad or other input means on the device to set the intensity level of the stimulation such as electric pulses, and current feedback to regulate the microprocessor output to voltage converter and current source to generate the stimulate pulses at the current level set by the keypad. The voltage converter converts battery voltage to a target voltage to drive the stimulation output. The output of the current source is connected to the electrodes, which contact skin. The batteries such as a rechargeable battery pack supply power to every block.

Similarly, visible light/IR and/or sound/vibration and/or heating stimulation generating circuit can also be incorporated into the device and controlled by a microprocessor, which is further controlled by keypad or remote control means such as cell phone app. The stimulation end (e.g. electrode, speaker, LED) can be built within the main device body with other components or separate with the main body containing other component and connected to the main body with wire and/or optical fibers to provide better portability and user convenience.

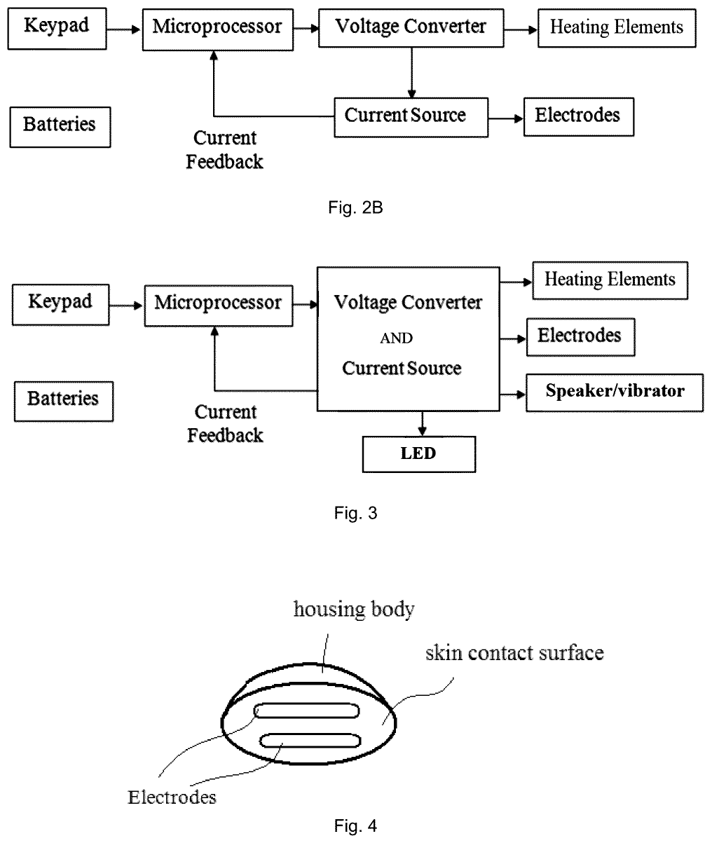

FIG. 2B illustrates an example of the circuit diagram suitable for the device with additional heating elements controlled by microprocessor and voltage converter. The voltage converter provide two voltages to dive the heating elements and electrodes. FIG. 3 illustrates an example of the circuit diagram suitable for the device with electrodes, heating elements, light emitting LEDs and sound/vibration generating speaker controlled by microprocessor. FIG. 3 in US patent application Ser. No. 16/819,165 from the current inventor illustrates an example of the circuit diagram.

FIG. 4 shows one embodiment of the device electrode assembly. On the skin-contacting surface of the device body, two electrodes are aligned in a side by side manner. One electrode function as cathode (or connects to the hot wire of the current source) and another electrode functions as anode (or connects to the neutral wire). External electrode can also be used to be connected with the housing via connection means such as wire to deliver the electrical pulse to the external electrode. If the device contains multiple housing bodies, each house body can contain only one electrode; the electrode in some housing function as cathode and the electrode in other housing function anode and they can change polarity during stimulation.

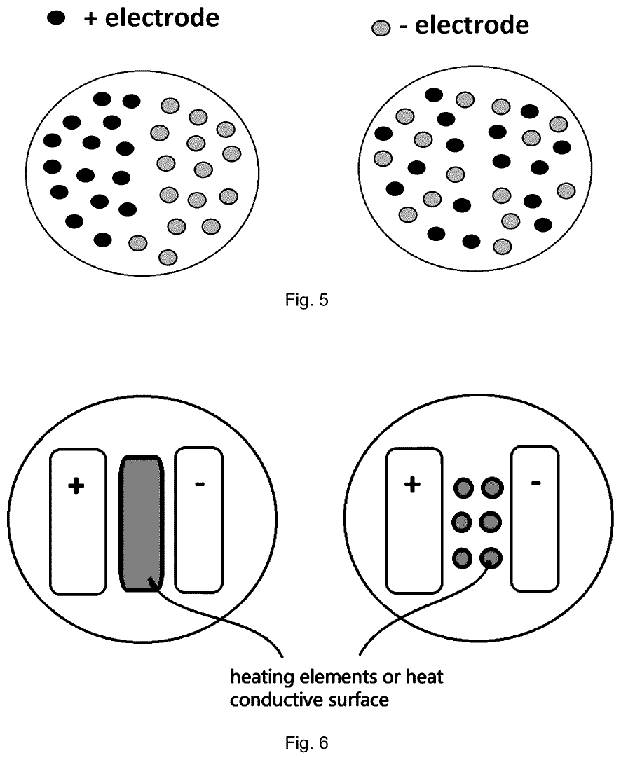

FIG. 5 shows skin contact surface of embodiments of another type of device electrode assembly. On the skin-contacting surface of the device body, multiple small electrodes are attached. Some electrodes function as cathodes and others function as anodes.

FIG. 6 shows embodiments of a device electrode assembly with heating element. On the skin-contacting surface of the device body, two electrodes are attached. Between the two electrodes is a heating element or a heat conducting area (e.g. a metal piece) with a heating element attached beneath. Alternatively, the heating element or the heat conductive area can be in an array form. Yet in another alternative, the heat conductive area can also be the electrode.

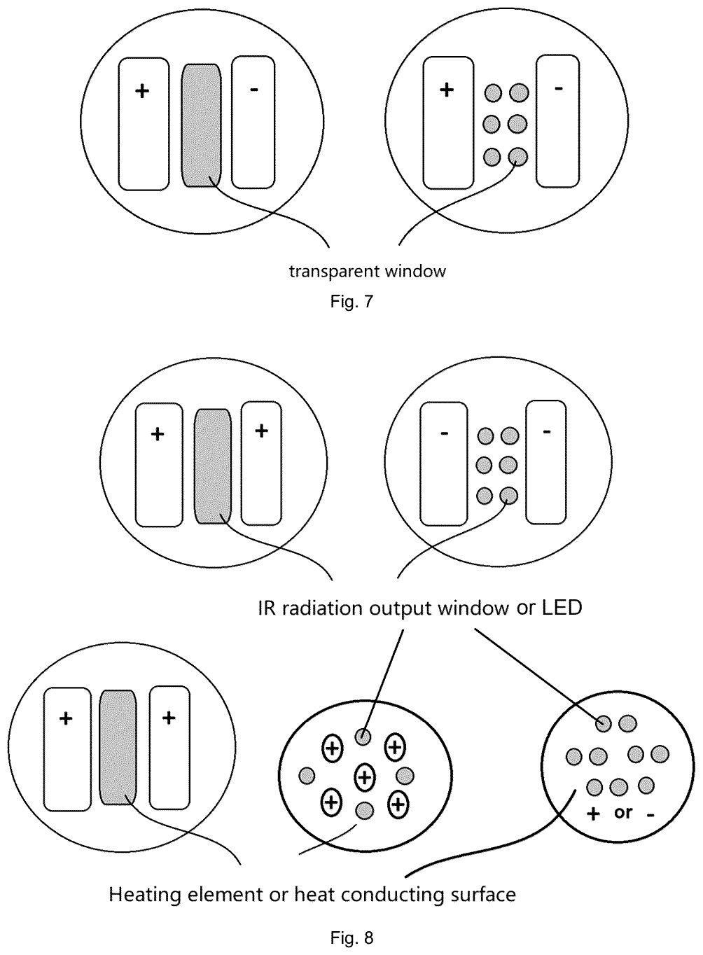

FIG. 7 shows embodiments of device electrode assembly with light radiation element. On the skin-contacting surface of the assembly, two electrodes are attached. Between the two electrodes is a transparent window with a laser or other type of light (e.g. LED) output source incorporated beneath. Alternatively, the transparent window can be in an array form. A laser or LED light source delivers the light to these windows with optical fibers. It can also has multiple LED embedded on the skin contacting surface.

Because multiple stimulating sites are used, some electrode assembly can function as cathode and others can function as anode, therefore no need to have different type of electrode (cathode or anode) integrated in one electrode assembly. When the electrodes are attached to the head's brain area, it can provide transcranial electrical stimulation, a noninvasive brain stimulation technique that passes an electrical current through the cortex of the brain. The electrical current is applied to an individual's scalp via two or more electrodes, and the current is conducted between electrodes through soft tissue and skull and a portion of the current penetrates the scalp/nearby area and can be conducted through the brain. In some embodiments said transcranial electrical stimulation is gamma wave stimulation and/or delta wave stimulation. The electrodes can be either cathode or anode or switch alternatingly. The electrode can also be attached to other body part as desired.

FIG. 8 shows embodiments of device electrode assembly with light radiation element and heating element. Its skin contacting surface has electrodes, light radiation window or LED, and heating elements or heating conducting surface with heating elements built underneath. The heating conducting surface can be electrode surface.

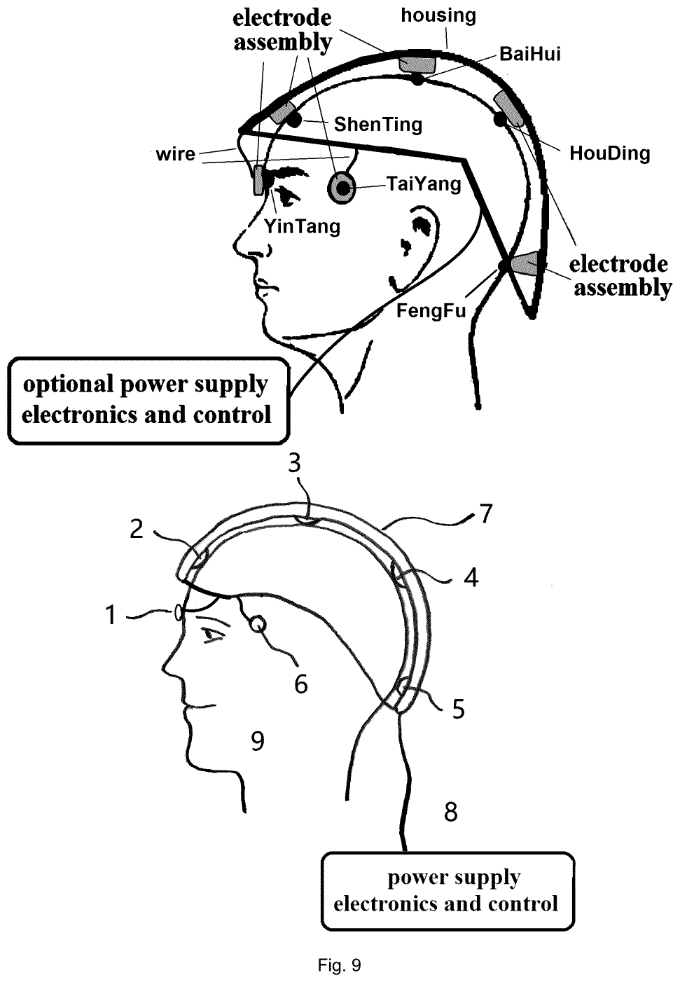

FIG. 9 is a perspective illustration of an embodiment of the device of the invention as worn on the head of a person using the device. The power supply and pulse generating circuitry for the device can be integrated within a helmet or hat like housing. Alternatively, the control, power supply and/or pulse generating circuitry can also be placed in another housing which is connected with the helmet or hat like housing with wires. The housing can also be in other shape and format such as a head band, as long as it contains means to affix it on the head or provide a support to place the housing on the head. An external control unit can be connected to the housing in the drawing, having stimulating power level control buttons and one status display (e.g. a LCD or LED display) or a touch screen control. The power level control buttons control the output power level of the electrodes. Higher power level (the intensity of the stimulation current) generally gives stronger stimulation, which may generate better therapeutic effects. The person in need use the control buttons to adjust the power level to achieve desired therapeutic effects and the best comfort. These buttons may also be used to control the pulse patterns. The LCD display displays the working status of the device such as the current power level and pulse pattern. A timing function can also be incorporated within the LCD.

The power supply and pulse generating circuitry for the device in FIG. 9, can be integrated within a helmet or hat like housing 7. Alternatively, the control, power supply and/or pulse generating circuitry can also be placed in another housing 8 which is connected with the housing 7 with wires as shown in FIG. 9. The housing can also be in other shape and format such as a head band, as long as it contains means to affix it on the head or provide a support to place the housing on the head. An external control unit 8 is connected to the housing in the drawings, having stimulating power level control buttons and one LCD status display. The power level control buttons control the output power level of the electrodes. Higher power level (the intensity of the stimulation current) generally gives stronger stimulation, which may generate better therapeutic effects. The patients use the control buttons to adjust the power level to achieve desired therapeutic effects and the best comfort. These buttons may also be used to control the pulse patterns. The LCD display displays the working status of the device such as the current power level and pulse pattern. A timing function can also be incorporated within the LCD. Four electrode assemblies 2, 3, 4 and 5 are aligned at the inner surface of the housing, allowing them to contact the skin at ShenTing, Baihui, HouDing and FengFu acupuncture sites respectively. Two external electrode assemblies 1 and 6 (such as those described in FIG. 4) are connected to the housing 7 with wire to get electricity and are attached to the sites TaiYang and YinTang sites respectively to provide stimulation. The housing sends out electrical pulse to the electrode assembly to apply electrical stimulation and/or other stimulation (e.g. light, sound stimulation) to the person in need. Self-adhesive electrodes could be used in external electrode assembly. A rigid or semi rigid connection structure containing the electric wire can also be used to link the external electrode assembly with the housing besides using wires.

The device in FIG. 9 can also contains means to provide heating, visible light/IR radiation, mechanical stimulation to skin on top of skull, acupuncture sites and/or their nearby area as previously described. The heating/light/IR stimulation can be applied at the same time with electric stimulation or be applied alternately or sequentially. The device can contain a sound/vibration generating means such as one or more speaker/vibrator, which can be attached to place close to said acupuncture sites or other head surface area (e.g. scalp or forehead or temple area) or placed into the ear in the form of speaker/earbud. As previously described, the means to generate light/sound/vibration (e.g. LED, speaker) can be integrated in the electrode assembly. In some embodiments the device contains sound/vibration and/or heating and/or visible light and/or IR radiation means only without any electric stimulating means and the therapeutic effect is achieved by direct heating and/or light radiation and/or sound only.

As shown in FIG. 9, a person in need places the device on his head and attaches the electrodes on the selected acupuncture points to apply electrical stimulation to these points through these electrodes. Heating and/or light radiation and/or sound/vibration stimulation can also be applied at the same time or separately to these sites using the heat/light/sound/vibration generating means built within the device. The primary stimulation points (some shown in FIG. 10) can be selected from Baihui, Sishencong, Fengchi, Shenting, Benshen, Shangxing, Fengfu, Yintang, Taiyang, Yamen, Dangyang, Dingshen, Shoumian, Chengling and Naohu acupuncture sites. One or more stimulation points can be used. Other adjunctive acupuncture points can be selected from Dazhui, NeiGuan, SanYinJiao, Renzhong, Shenmen, Xinyu, Jianshi, Guanyuan, Yongquan, Taixi, Xuehai, Shuangling, Wangu, Dashu, Renying and Shenyu acupuncture sites. Using adjunctive acupuncture points in combination with primary stimulation points gives higher effectiveness than using the primary acupuncture points alone. In addition, stimulating some of these points requires attaching the electrode or other stimulation generating means in places other than the head (e.g. electrode or light or vibration need to be attached to the wrist to stimulate NeiGuan point). In order to stimulate adjunctive acupuncture points, external electrodes or other stimulation output end connected with the main housing (e.g. the housing on the head or the external power supply/electronics) can be used to reach these sites. The stimulation at each point/area can be simultaneously or sequentially based on a predefined pattern (e.g. cycle from left to right or from center to peripheral and etc.). As described previously, the electrical stimulation can be replaced with heating or light such as red light/IR radiation or sound/vibration with the corresponding means.

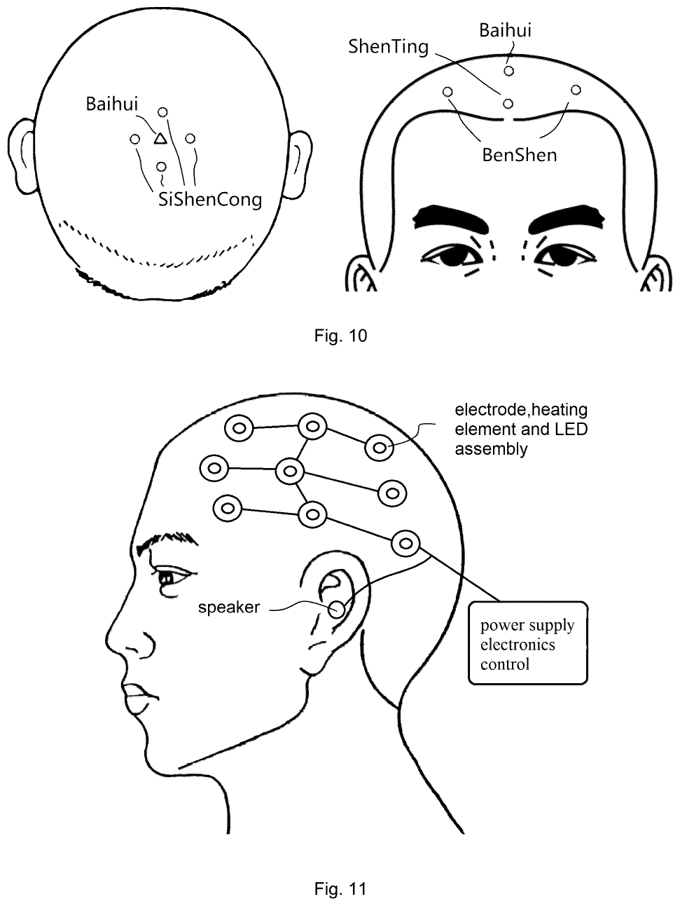

Alternatively, the area for stimulation is not limited to acupuncture sites. It can be placed on other areas on the head as long as the subject feel comfortable. In some embodiments, the device as shown in FIG. 9 or 11 can have an array of electrodes or an array of electrodes with heating/light radiation and/or sound/vibration means covering the skin on top of the brain with the stimulation pulse frequency between 25˜100 Hz, preferably between 35˜45 Hz and/or delta wave frequency. In some embodiments, the device produces said stimulation pulse frequency at delta wave (0.1-4 Hz such as 0.5-4 Hz, e.g. 1 Hz). In some embodiments, the device produces said stimulation pulse frequency at delta wave frequency at some time point and gamma wave (35˜45 Hz) at other time point. For example, a device used by Novocure can be modified to give electrical, LED light and vibration stimulation with a frequency of 40 Hz and delta wave frequency for improving brain function and treating senile dementia including Alzheimer's disease. The device disclosed in US patent numbers U.S. Pat. Nos. 8,715,203, 7,805,201 can be modified to have the desired frequency for the current invention.

In example 1, a device described in FIG. 9 or 11 or 13-17 is attached to a volunteer's head to stimulate the skin on top of skull or the acupuncture sites selected from YinTang, ShenTing, BaiHui, TaiYang and SiShengCong or is attached to other body part to stimulate skin surface there, with combination of electrical pulse, light pulse (e.g. 670 nm and 810 nm) of 40 Hz at the power level of the volunteer feels comfortable for ˜0.5 hour every day to improve brain function. An optional sound/vibration pulse at gamma wave frequency (35-50 Hz) using a speaker of the device attached to ear or skull or other body part can also be applied at the same time at the power level of the volunteer feels comfortable. This method and device can also be used to improve learning during study and improving reasoning and problem solving capacity. A device can also have external LED light that face the open of nose channel or be inserted into nose channel to provide the same frequency light stimulation and the user can use it in combination with the light/sound/electric stimulation to the skull to improve brain function and improve learning efficacy during study. Visible light pulse from a light source (e.g. LED) at gamma wave frequency (35-50 Hz) can also be applied to the eye of the person or environment at the same time or sequentially. A device can also have an external LED light and electrode that can be inserted into ear duct to provide the same frequency light/electric stimulation and the user can use it in combination with the light/electric/sound stimulation to the skull to improve brain function and improve learning efficacy during study and improve reasoning and problem solving. In another example, the frequency of stimulation of the device and method in example 1 is 40 Hz.

In one study, a volunteer receives electrical pulse from electrodes attached to the skin surface on top of the skull at non-acupuncture sites, red light/NIR pulse (630 nm and 850 nm) radiation and vibration stimulation to the skin on top of skull all at a frequency between 35˜45 Hz with power level the volunteer feels comfort for 0.5-1 hour daily. Different stimulation has the same frequency and the onset of the 35˜45 Hz pulse in different stimulation are aligned to the same time point. The volunteer feels improved memory, sleep quality and cognitive activity.

In another study, a volunteer receives electrical pulse from electrodes of 40 Hz, heat stimulation from heating elements, continuous red light/NIR (660 nm and 880 nm) radiation to stimulate the acupuncture sites selected from TaiYang, YinTang, BenShen, HouDing, FengDu and FengChi, and sound stimulation pulsed at 40 Hz using a speaker in the ear at power level the volunteer feels comfort for 0.5-1 hour every day for 2 weeks. The onset of the 40 Hz pulse in electrical and sound stimulation are aligned at the same time point. The volunteer feels improved memory, sleep quality and cognitive activity.

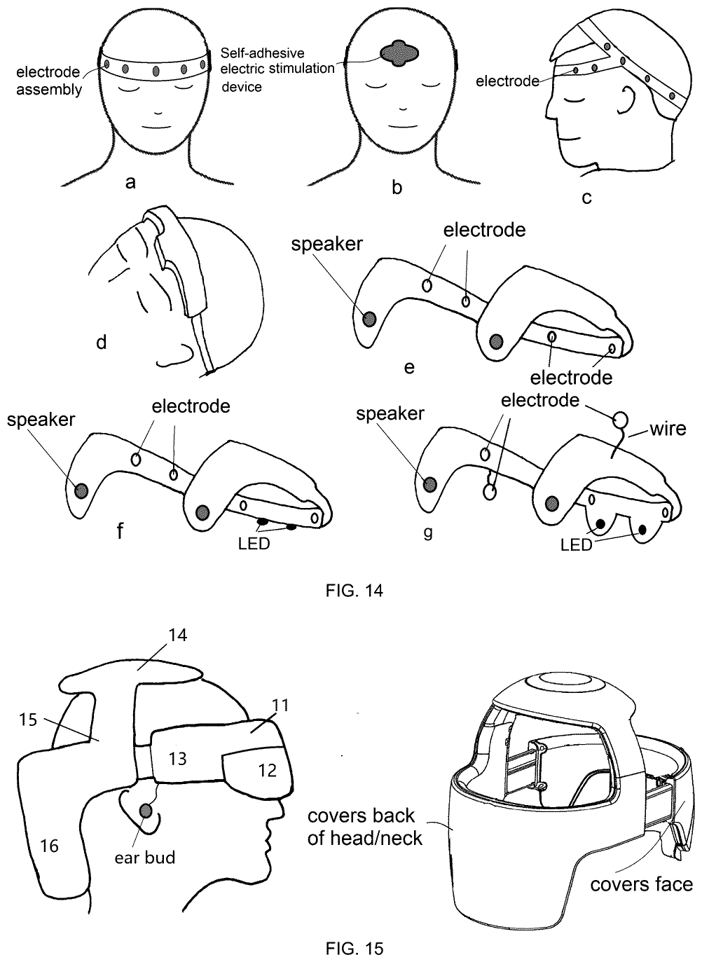

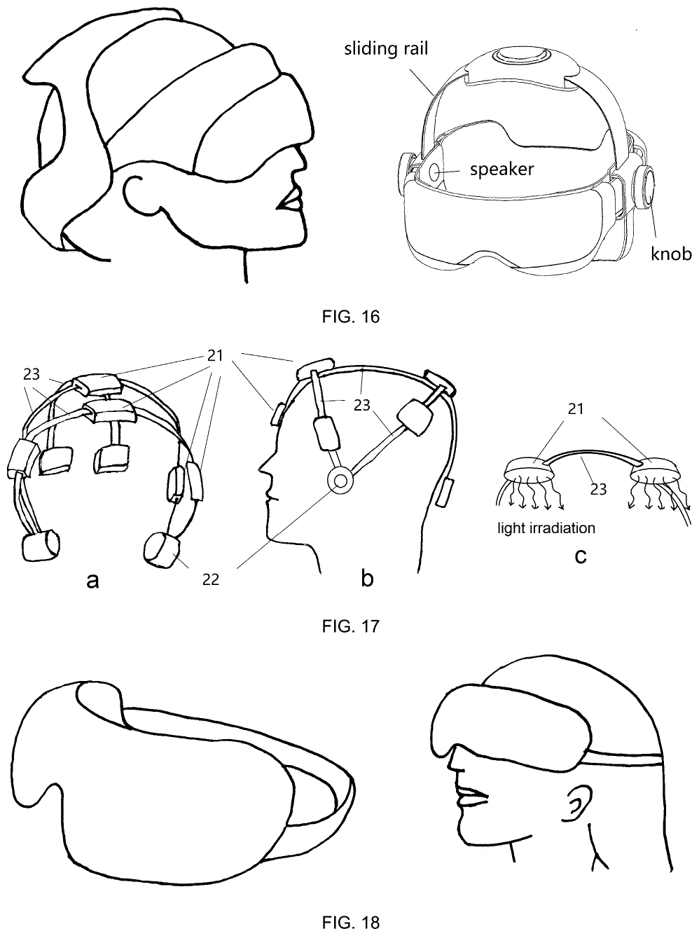

In example 2, the device and method are similar to the device and method of example 1 except only gamma wave frequency (35-50 Hz) is employed in example 1 while both gamma wave frequency and delta wave frequency stimulations are used in example 2. Besides gamma wave frequency stimulations as that in example 1, the device according to example 1 further comprise means that can provide delta wave frequency (0.1-4 Hz such as 0.5-4 Hz) stimulation selected from light/sound/vibration/electric stimulation; and the method according to example 1 further use delta wave frequency (0.1-4 Hz such as 0.5-4 Hz) frequency of light/sound/vibration/electric stimulation to the subject in need to improve brain function and improve learning efficacy during study and improve reasoning, thinking ability and problem solving capability. The gamma wave frequency and delta wave frequency stimulations are used sequentially at different timepoint each day. For example, gamma wave frequency stimulations are applied during daytime or before/during sleep; and delta wave frequency stimulations are applied in the night or during sleep such as during slow wave sleep period. For example, a device described in FIG. 9 or 11 or 13-17 is attached to a volunteer's head to stimulate the acupuncture sites selected from YinTang, ShenTing, BaiHui, TaiYang and SiShengCong or is attached to other body part to stimulate skin surface there, with combination of electrical pulse, light pulse (e.g. 670 nm and 810 nm) of delta wave frequency at the power level of the volunteer feels comfortable for 0.5-2 hour in the night or during sleep such as during slow wave sleep period every day to improve brain function. Visible light pulse from a light source (e.g. LED) at delta wave frequency (0.1-4 Hz such as 0.5-4 Hz) can also be applied to the eye of the person or environment at the same time or sequentially. An optional sound/vibration pulse at delta wave frequency (0.1-4 Hz such as 0.5-4 Hz) using a speaker or vibrator of the device attached to ear or skull can also be applied at the same time at the power level of the volunteer feels comfortable. This method and device can also be used to improve learning during study. A device can also have external light source (e.g. LED) that face the open of nose channel or be inserted into nose channel to provide the same delta wave frequency light stimulation and the user can use it in combination with the light/sound/electric stimulation to the skull or other body part to improve brain function and improve learning efficacy during study. A device can also have an external light source (e.g. LED) and electrode that can be inserted into ear duct to provide the same electrical pulse, light pulse (e.g. visible light or red light/NIR such as 670 nm and 810 nm light) frequency light/electric stimulation and the user can use it in combination with the light/electric/sound/vibration stimulation to the skull to improve brain function and improve learning efficacy during study and improve reasoning, thinking activity and problem solving capability. In another example, the delta wave frequency of stimulation of the device and method is between 0.1-1 Hz. The device can be in one integrated structure that provide both gamma wave stimulation and delta wave stimulation, or contains a part that can be detached from the main body to provide delta wave stimulation. Alternatively the device is essentially a kit, that contains two separate components that can work separately, e.g. one helmet format (e.g. those in FIG. 15-16) component to provide gamma wave stimulation during and another separate component (e.g. those in FIG. 13-14) to provide delta wave stimulation.

In example 3, the device and method of example 1 or 2 is to stimulate ShenTing, BaiHui, TaiYang and SiShengCong sites. In another example, the stimulation in device and method of example 1 or 2 is applied to TaiYang or temple area only. In another example, the stimulation in device and method of example 1 or 2 is applied to skin area on top of skull. In another example, the stimulation in device and method of example 1 or 2 is applied to skin area on other body part such as wrist, waist, arm, hand, palm, neck, head, face, forehead, hand, feet, finger, toe, palm or sole.

In one study, a volunteer receives electrical pulse from electrodes attached to the skin surface on top of the skull at non-acupuncture sites, red light/NIR pulse (630 nm and 850 nm) radiation and vibration stimulation to the skin on top of skull all at a frequency between 0.5-4 Hz with power level the volunteer feels comfort for 0.5-1 hour every day. Different stimulation has the same frequency and the onset of the 0.5-4 Hz pulse in different stimulation are aligned to the same time point. The volunteer feels improved memory, sleep quality and cognitive activity.

In another study, a volunteer receives electrical pulse from electrodes of 1 Hz, heat stimulation from heating elements, continuous red light/NIR (660 nm and 880 nm) radiation to stimulate the acupuncture sites selected from YinTang, BenShen, HouDing, FengDu and FengChi, and audio sound stimulation pulsed at 1 Hz using a speaker in the ear at power level the volunteer feels comfort for 0.5-1 hour every day. The onset of the 1 Hz pulse in electrical and sound stimulation are aligned at the same time point. The volunteer feels improved memory, sleep quality and cognitive activity.

In another study, a volunteer receives electrical pulse from electrodes of 2 Hz to TaiYang acupuncture site. Additional 2 Hz light at green light wavelength and 880 nm as well as audio sound pulsed at 2 Hz from a speaker are provided as environmental light/sound stimulation without physical contact with the volunteer. The onset of the 2 Hz pulse in electrical, light and sound stimulation are aligned at the same time point. The stimulations are given during sleep at power level the volunteer feels comfort for 0.5-1 hour every day. The volunteer feels improved memory, sleep quality and cognitive activity.

In another study, a volunteer receives 0.1-1 Hz red light well as sound of 100 Hz pulsed at 0.4-1 Hz from a speaker as environmental light/sound stimulation without physical contact with the volunteer. The light and sound pulse has the same frequency between 0.1-1 Hz and the onset of the pulse in light and sound stimulation are aligned at the same time point. The stimulations are given during sleep for 1 hour every day at a stimulation level that does not wake up volunteer. The volunteer feels improved memory, sleep quality and cognitive activity.

In example 4, a device containing of stimulation output means such as those described in FIG. 11-17 is attached to a volunteer's head to stimulate the skin surface of head at either acupuncture sites or non-acupuncture sites, or is attached to other body part to stimulate skin surface there, with electrical pulse, red light and/or NIR pulse (e.g. 655 nm, 810 nm and 850 nm), sound/vibration stimulations at gamma wave frequency (35-50 Hz) and delta wave frequency (0.1-4 Hz) at the power level of the volunteer feels comfortable for 0.5-2 hours every day for 2 weeks to improve brain function (such as memory, sleep quality and cognitive activity) and to improve learning efficacy and sensory processing and improve reasoning, thinking activity and problem solving capability. The gamma wave frequency and delta wave frequency stimulations are used separately at different timepoint each day. For example, gamma wave frequency stimulations are applied during daytime and delta wave frequency stimulations are applied in the evening or during sleep. In another example, gamma wave frequency stimulations are applied in the evening or during sleep and delta wave frequency stimulations are applied during daytime or during awake state. The stimulations can be applied to head surface areas covering frontal lobe only or parietal lobe only or occipital lobe only or temporal lobe only or any of their combinations. In another example, the device and method of example 4 further comprising heating elements attached to a volunteer's head to stimulate the acupuncture sites selected from YinTang, BenShen, HouDing, FengDu and FengChi or non-acupuncture site on scalp together with other type of stimulations. The device shown in FIG. 11 has 18 stimulation output ends (9 on each side of head), in some embodiments less (e.g. 4-10) or more (e.g. 20-30) can be incorporated in the device and used by said method to apply electric and/or light and/or sound/vibration stimulations to head. Those stimulation output ends can be spatially evenly arranged or arranged in certain configurations to apply stimulations to certain area. Furthermore to improve brain function, the array of said stimulation output end can be embedded in the skin contact side of a hat or helmet type covering that can cover the head to provide red light and/or NIR stimulation to the head surface. The hat or helmet type covering can have a light reflecting layer to prevent light leaking and keep most of the light towards the skin. The red light and/or NIR stimulation frequency can be gamma wave (35˜45 Hz), alpha wave (7.5-14 Hz), theta wave (4-7.5 Hz), delta wave (0.1-4 Hz) at different time point (e.g. each frequency for 10 min) and can also be continuous non-pulse stimulation (e.g. for 10 min). An assay of heating elements can also be embedded in the hat or helmet type covering to provide heat stimulation to brain area.

In one study, a volunteer receives electrical pulse at a frequency between 35˜45 Hz from electrodes to stimulate the acupuncture sites TaiYang, YinTang, BenShen, HouDing, FengDu and FengChi, continuous red light/NIR pulse (630 nm and 850 nm) radiation to the skin on top of skull, and sound stimulation of audio tone pulsed at a frequency between 35˜45 Hz from a speaker to ears for 0.5-1 hour every day during daytime at power level the volunteer feels comfort. Different pulsed stimulation has the same frequency and the onset of the 35˜45 Hz pulse in electrical and sound stimulation are aligned to the same time point. The volunteer also receives electrical pulse from electrodes of 1-2 Hz to TaiYang acupuncture site, additional 1-2 Hz light at green light wavelength and 200 Hz audio sound pulsed at 1-2 Hz from a speaker as environmental light/sound stimulation without physical contact with the volunteer for 1-2 hour every day during sleep. Different stimulation has the same frequency and the onset of the pulse in electrical, light and sound stimulation are aligned to the same time point. The volunteer feels improved memory, sleep quality and cognitive activity and the improvement is more significant than using stimulation during daytime or during sleep only.

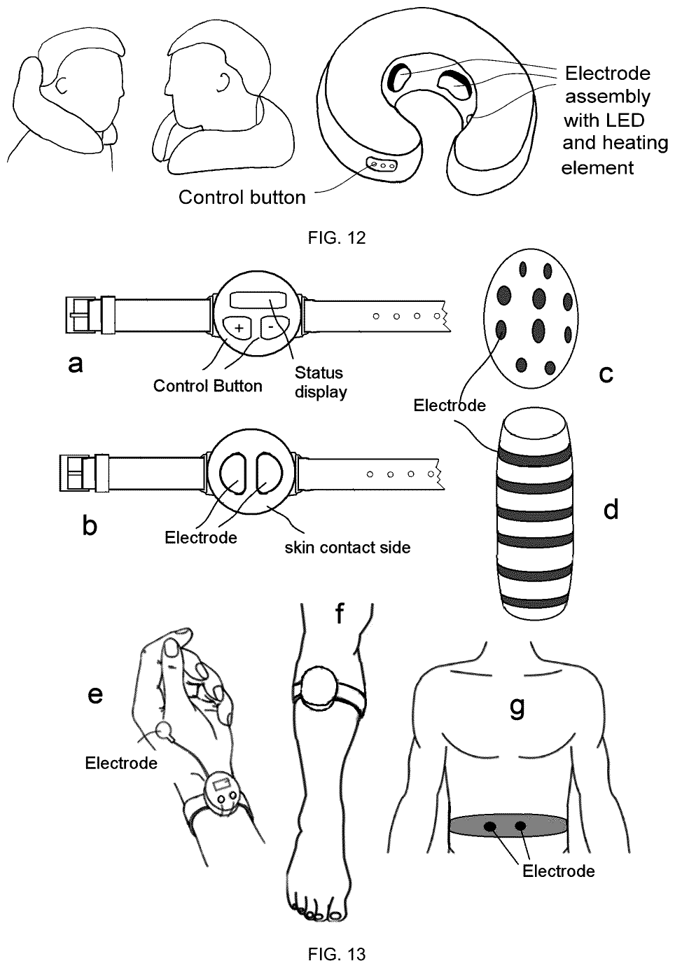

A device with configuration/shape similar to that in FIG. 9 or those in examples 1-4 containing means to provide heating (e.g. using heating element or thermal IR radiation) to the head, especially the top and backside of the head can also be used to improve brain function and/or reduce symptoms of dementia, by improving the blood flow in the brain. Higher temperature (heating) to the brain area can boost the blood flow, therefore improve brain function and/or reduce symptoms of dementia. This device is essentially a hat or helmet with built-in heating means that can cover most of the area on the top and/or back of the head, not limited to the acupuncture sites previously described. It can have an insulating layer to maintain the high temperature inside the covered area (e.g. 40˜60 C°). The heating can be constant or intermittent (e.g. one minute on 30 seconds off alternately). People in need can put it on to heat their brain area to improve brain function and/or reduce symptoms of dementia (e.g. 0.5 hr 3 times a day). The heating stimulation can be applied at the same time as other stimulation or be applied alternately or sequentially. In some embodiments the device contains heating means and light radiation at gamma wave frequency and delta wave frequency stimulations means only and the therapeutic effect is achieved by heating and/or light radiation. The device can also have an attachment with built in heating and/or light stimulation means that can wrap the neck of the person in need to heat their blood vessel in the neck to boost the blood flow and provide red light/IR irradiation as well, which can be essentially a muffler/scarf with built in heating/light stimulation means. Another format of device is configured to wrap the neck of the person only without being attached to a hat/helmet like configuration. FIG. 12 shows exemplary format of said device with a shape configured to be wrapped around neck. FIG. 12 left shows a device configuration with an extension at the back that can reach or close to the lower end of occipital region, which contains heating elements and/or red light/NIR stimulating means (e.g. 670 nm and 810 nm LEDs) positioned to apply heating effect and/or light stimulation to carotid artery and jugular vein area, and optionally electrodes to apply electric pulse to Fengchi and/or Fengfu and/or Renying acupuncture point area and/or carotid sinus area. It can also be used to reduce blood pressure by using said device to apply heat (e.g. 40˜60 C), light and/or electric stimulation to the neck area for 5˜20 min or until desired effect is achieved. The current invention discloses a method to improve brain function and/or reduce symptoms of dementia by increase the blood flow in the brain and activate blood cell mitochondrial activity, which is achieved by applying heating and light irradiation (continued or gamma wave frequency and/or delta wave frequency) to neck and/or head region of the person in need.

Vibration/sound stimulation with the frequency selected from of gamma wave (35˜45 Hz), alpha wave (7.5-14 Hz), theta wave (4-7.5 Hz), delta wave (0.1-4 Hz) or their combinations can be applied to the subject in need for the above indications to improve brain function and the corresponding means (e.g. speaker, motor, vibrator) can also be placed in the house of the said above devices such as those in examples 1-4 and FIGS. 9-26. The sound and vibration can also be a mimic of cat purr which is normally from 20 Hz to 150 Hz. The housing can also be a pillow type shape that a user can lay down and place the head on top of it to receive said vibration and/or sound stimulations. In some embodiments the pillow shape device produces vibration/sound stimulation. In some embodiments the pillow shape device produces vibration/sound stimulation and heating stimulation and cooling stimulation. In some embodiments the pillow shape device produces vibration/sound stimulation and light stimulation with optional heat stimulation.

In some embodiments the electrical and/or sound/vibration or light pulse selected from gamma wave (35˜45 Hz), alpha wave (7.5-14 Hz), theta wave (4-7.5 Hz), delta wave (0.1-4 Hz) or their combinations is applied to area other than head, such as wrist, waist, leg, foot, arm, palm and finger. The frequency can be either apparent frequency (not modulated) or FIMF frequency or AMF frequency or PWM frequency. The device can be a wrist band or watch type device or hand holdable format device that have a skin contact electrode on the house, such as those described in patent application US20040102819 and china patent CN2676947Y. It can also have a configuration that can be hold in the palm such as an egg shape so the stimulation can be applied to the palm. Examples of device configurations can be used are shown in the FIG. 13. FIGS. 13c and d show devices that can be hold with hand with electrodes in small circle shape or belt shape on their surface to stimulate palm. Additional sound/vibration and/or heating/cooling stimulation can also be provided by the hand hold device with means incorporated inside. For example, red light/NIR LEDs can be embedded on the surface of the handhold device and speaker/vibrator can be placed inside the device. The hand hold device can be set to emit gamma wave stimulations when user is awake or emit delta wave stimulations when user is in sleep to improve brain function. In one example, a user holds a hand hold device when he is awake to receive electric pulse of 40 Hz to his palm as well as a vibration stimulation to his palm at 40 Hz and a red light stimulation to his palm at 40 Hz 0.5-1 hr every day, where the onset of 40 Hz pulse in different stimulation are aligned to the same time point; the user also holds the hand hold device before and during sleep to receive electric pulse of 0.5-1 Hz to his palm as well as a vibration stimulation to his palm at 0.5-1 Hz and an audio sound pulsed at 0.5-1 Hz 1-3 hours every day, where different type of stimulation has the same frequency and the onset of 0.5-1 Hz pulse in different stimulation are aligned to the same time point; to improve the user's brain functions and improve sleep quality. FIG. 13g shows a waist belt shape device that can provide stimulation to waist or abdomen area. The user can use those devices 10-30 min 1-3 times a day to improve brain function. The user can also use them to improve learning during study, for example, by applying 35˜45 Hz gamma wave stimulation (electric or electric+sound/vibration) during study time to improve sensory processing and learning efficacy and improve reasoning, thinking activity and problem solving capability. Multiple different type of devices can be used concurrently to stimulate multiple area of the body to achieve better effect, where the stimulation pulse need to be synchronized so they use the same frequency and the onset of the pulses in different stimulation are aligned to the same time point.

As described, the devices described in the current invention to generate gamma wave stimulations (such as electric pulse, visible light/NIR, sound/vibration) including gamma wave stimulations can also incorporate means to produce stimulations having frequencies selected from alpha (7.5-14 Hz), theta (4-7.5 Hz) and delta (0.1-4 Hz) wave or their combinations. Therefore the method to improve brain function and treating senile dementia including Alzheimer's disease further include giving a subject in need with the combination of gamma wave stimulations and stimulations selected from alpha (7.5-14 Hz), theta (4-7.5 Hz) and delta (0.1-4 Hz such as 0.5-4 Hz) wave stimulation. In some embodiments, the method to improve brain function and treating senile dementia including Alzheimer's disease further include giving a subject in need with the combination of stimulations of with frequency of gamma wave and delta (0.1-4 Hz such as 1-2 Hz) wave. The stimulation can be electric pulse, light, NIR, sound/vibration and their combinations. In one example, the person's head such as the acupuncture points described previously is given electric stimulation of gamma wave frequency (35˜45 Hz) for 30-60 min and then alpha or theta or delta, or alpha and then theta wave frequency for 30-60 min before sleep as well as delta wave frequency ((0.1-4 Hz such as 1-2 Hz) stimulation 30 min several times during sleep.

The device suitable for the current invention can be in many different formats, configurations and shapes such as in the form of hat or hairband or glass or helmet or a self-stick patch form. FIGS. 14-17 show some exemplary formats/configurations that can be attached to head. Their skin facing portion/skin contacting surface can contain one or more electrodes that can be used to apply electric pulse to the subject in need. It can also include a speaker or motor (vibrator) to give sound/vibration stimulations as well as light/NIR output and/or heating elements such as LED. They can have a means to commutate with a remote controlling device such as cell phone with either Bluetooth or Wi-Fi or other radio frequency signals and be controlled by the remote controlling device. A cell phone application can be used to control the device.

As shown in FIG. 14a, electrodes in a head band type fixture are attached to the head to provide gamma wave (35˜45 Hz) and delta wave (0.1-4 Hz) frequency electric stimulation to improve brain function, it can also be used to improve learning during study and improve reasoning, thinking activity and problem solving capability. FIG. 14b shows another format in which the stimulation output end such as electrode, light and sound/vibration stimulation means is located on the skin contact side of the device and attached to the forehead using self-stick patch. FIG. 14c shows another format in which the stimulation output end is attached to the skin contact side of the device. FIG. 14d shows another format in which the LEDs and electrodes of the device is attached to the temple area; and another LED and speaker/vibrator is mounted to the forehead area.

FIG. 14e shows another format similar to a glass frame without lens area in which one or more electrodes is located on the skin contact side of the device. The device can be mounted to the forehead and speakers is mounted close to the ear area, attached to the head in a configuration similar to that in FIG. 14d or similar to wearing a glass. The device contains built-in electronics to produce 30-50 Hz electric pulse stimulation in combination with 30-50 Hz audio sound pulse stimulation to a user; it can also produce 0.1-2 Hz electric pulse stimulation in combination with 0.1-2 Hz audio sound pulse stimulation to a user at a different time. The device can communicate with a cell phone, which can control the device to adjust its stimulation frequency, intensity and running time. FIG. 14f is a device similar to that in FIG. 14e with additional LEDs facing eye directions to provide light stimulation to the eyes in either gamma wave frequency or delta wave frequency. The light can be either white light or colored light such as yellow light or green light or red light or NIR or their combinations. FIG. 14g is a device similar to that in FIG. 14f except it has a glass shape with two LEDs are placed in the area in front of the eye to provide light irradiation, and two or more external electrodes attached to the main body with flexible wire to provide stimulation to more skin areas. It can has an eye mask/eye shield shape similar to those in 18 and 19.

FIGS. 15 and 16 show helmet like device configurations that has means to provide stimulation to head region. Parts 14 and 15 have light source (e.g. LEDs) and electrodes on their skin contacting side to provide electric pulse and red light and/or NIR light (e.g. 670 nm and 810 nm at 20-200 mW/cm²) pulse stimulations to the skin area covering the parietal lobe and occipital lobe. Part 16 has LEDs and electrodes to provide electric pulse and red light/NIR light pulse stimulations to the skin area covering occipital lobe and Fengchi, Fengfu acupuncture sites. Parts 11, 13 have LEDs, electrodes and optional speakers/vibrators to provide electric pulse, light pulse and sound/vibration stimulations to skin areas covering frontal lobe and temple area. The frequency of stimulations is selected from of gamma wave (35˜45 Hz), alpha wave (7.5-14 Hz), theta wave (4-7.5 Hz), delta wave (0.1-4 Hz) or their combinations. Part 12 contains a light source such as LED to provide light stimulations to eyes (either closed eye through eyelid or opened eye) at gamma wave or delta wave frequency at different time period; wherein the light can be either red light or NIR or white light or yellow or green light or their combinations. Additional heating/cooling elements as well as optional speakers/vibrators can also be incorporated within parts 11-16 to provide heating or cooling stimulation either continuously or intermittently. A pair of ear bud is connected to part 13 to provide gamma wave or delta wave sound pulse stimulation to the user. The helmet comprises two pieces, piece containing part 11-13 can be pushed closer to or moved away from piece containing part 14-16 to accommodate the different head size of the user. As shown in FIG. 16, knob and sliding rail are used to adjust the device to fit user's head. Vibrator such as airbag can be placed on the skin contact side of FIG. 16 to provide massage effect, e.g. at delta wave frequency.

FIG. 17 shows another types of device configuration that multiple stimulation output units 21 are mounted on sliding rails 23 to allow user to change stimulation sites. Stimulation output unit 21 has LED and electrode to provide gamma wave (35˜45 Hz) or delta wave (0.1-4 Hz) frequency stimulations. Speaker or vibrator 22 can provide gamma wave (35˜45 Hz) or delta wave (0.1-4 Hz) frequency stimulation to area close to ear. Additional LED that can provide light stimulation to nose can also be incorporated. FIG. 17c shows two stimulation output units 21 that can provide heat and NIR light stimulation to the area beneath once is mounted on head by sliding rail 23.

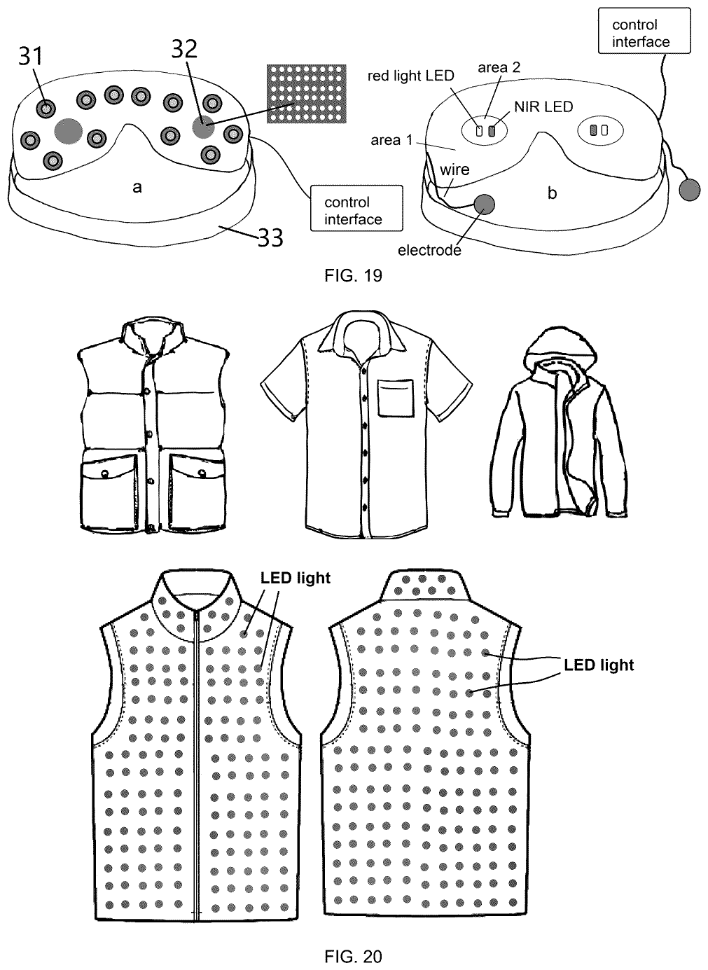

FIG. 18 shows another type of device having eye mask configuration that covers the eye and nearby area to provide both heat stimulation and continuous or pulsed light stimulation to improve brain function and eye health. The light can be white light or yellow light or green light or red light or NIR or their combinations at gamma wave (35˜45 Hz), alpha wave (7.5-14 Hz), theta wave (4-7.5 Hz), delta wave (0.1-4 Hz) frequency or their combinations. The light intensity needs to be comfortable to the user. In one example as shown in FIG. 19a, the device of FIG. 19a has multiple heating elements 31 built within, two LEDs under area 32 facing the eye, and elastic head band 33 to place it on the head. The eye facing surface 32 has many small holes to allow light from LED underneath to pass through to reach the eye. It can also be a transparent window to allow light to pass or a small hole with a diverging lens to allow light to irradiate a larger area after it pass the diverging lens. Optional electrodes can also be incorporated into the device to stimulate the areas around the eyes. As shown in FIG. 19b, two or more external electrodes can be attached to the device main body with flexible wire to provide stimulation to skin.

The devices in FIGS. 18 and 19 can be used to improve vision, treating dry eye, reduce eye inflammation, AMD and improve tissue and wounds repairing of the eye by providing visible light or IR light irradiation to eye lid (can be closed eyelid) and/or eye area as well as heating simulation to eye area with optional electric pulse stimulation as previous described. The light irradiation can be white light or red light or NIR or their combinations. The light stimulation can be either in the pulse frequency as that used to improve brain function, or work continuously without pulse, or intermittently (e.g. on for 2 min, off for 1 min). For example, the light source can be a white light LED or a 780 nm LED to provide 10-50 mW/cm² light at cornea. The light treatment can be performed 2-20 min 1-3 times a day. The heat treatment can be performed as frequent as needed. The light and heat stimulation can be applied simultaneously. The white light stimulation is suitable for closed eye stimulation to irradiate eye lid.

The devices shown in FIG. 19 are exemplary devices to improve vision, treating dry eye, reduce eye inflammation, AMD and improve tissue and wounds repairing of the eye by providing red light and/or NIR light irradiation to eye lid (can be closed eyelid) and/or eye area as well as heating and/or cooling simulation to eye area with optional electric pulse stimulation to eye area or the acupuncture sites that can improve eye health (e.g. Sizhukong, Taiyang, Sibai, Qingming, Cuanzhu and Chengqi) as described previously. The light irradiation can be red light or NIR or their combinations. It has a USB interface to connect to a power source such as a portable rechargeable battery bank to power the electric circuits, heating means and LED. It has a control interface to adjust stimulation pattern, intensity and time. FIG. 19b shows the eye facing side. It has a belt like head band to allow it be attached to the eye area. It has a soft fabric covered raised area 1 containing heating elements to provide heating stimulation at 40˜60° C. and sunken concave area 2 containing one or more 630 nm red light LED and one or more 850 nm NIR LED facing eye ball to provide light irradiation. Additional massage means such as vibrator (e.g. airbag) can also be incorporated. Red light/near infrared light (NIR) can activate mitochondrial function, increase ATP production and have deep tissue penetration (several centimeters). They can improve cell energy metabolism and improve/restore cell vitality. They can promote tissue, nerve growth and repair in wound healing. Irradiating fundus with NIR or red light can improve vision by improve photoreceptor sensitivity, which decreases after age 40, and can treat AMD in human subject. Applying heat to the eyes can relieve dry eye and eye fatigue. In one example, a volunteer use a FIG. 19b type device to receive heating stimulation at 40˜60° C. to eye area and red light/NIR (670 nm and 810 nm) stimulation to closed eye 5 min 3 time a day for a week, the volunteer feel improved eye vision, relief of eye fatigue and dry eye symptom.

The device in the current invention to improve brain function can also contain electroencephalography (EEG) sensor (electrodes) to detect the brain wave of the subject and to give stimulation accordingly to induce the desired brain wave in the subject. Different people may have different brain wave frequency, their brain wave frequency can be measured (e.g. their gamma wave and delta wave frequency) and the said device can use these specific brain wave frequencies for stimulation. For example, if one person's gamma wave frequency is normally 50 Hz, then a 50 Hz electric or light or sound stimulation can be applied to this person to improve his brain function. When the target brain wave frequency is significantly different from the current brain wave frequency, the device can gradually change the stimulation frequency towards the target frequency until the desired brain wave frequency is achieved. For example, if the target brain wave frequency is delta wave (e.g. 0.5-4 Hz) and the current measured brain wave frequency is beta wave (12 to 38 Hz), the device can apply alpha wave frequency (7.5-12 Hz) stimulations to the user until the measured brain wave is alpha wave, and then apply theta wave frequency (4-7.5 Hz) stimulations to the user until the measured brain wave is theta wave, and then apply delta wave frequency (0.5-4 Hz) stimulations to the user until the measured brain wave is delta wave and then maintain the delta wave stimulations for certain period of time.

Within each type of stimulations, the frequency can also be changed gradually towards the target frequency, for example, the current measured brain wave is 12 Hz alpha wave and the target is 2 Hz delta wave, then stimulation is applied gradually from 12 Hz to 7.5 Hz then gradually to 4 Hz and then gradually to 2 Hz. In some embodiments when delta wave (0.5-4 Hz) stimulations is to be applied during slow wave sleep period, the device can monitor the user's brain wave and once it is in the slow wave sleep mode, delta wave stimulation is applied or stimulation is applied gradually from current measure brain wave frequency (e.g. theta wave) to delta wave frequency.

The device can monitor the user's sleep period and apply stimulation accordingly. In some embodiments, the device applies gamma wave frequency stimulation in user's rapid eye movement sleep period to improve brain function.

In some embodiments, both gamma wave and delta wave stimulations can be applied either when the user is awake. In some embodiments, both gamma wave and delta wave stimulations can be applied when the user is in sleep. In some embodiments, gamma wave stimulation is applied when the user is awake and delta wave stimulation is applied when the user is in sleep.

When stimulation is applied to user in sleep, preferably the stimulation should not wake up the user. This can be done by adjust the intensity to a lower level and/or gradually and slowly increase and decrease the intensity when the stimulation pulse is applied.

The current invention also discloses method and device to improve body function, improve immunity, improve metabolism, improve skin condition, treat skin diseases, anti-aging, improve body's cellular mitochondrial activity, reduce weight, weight control, burn fat, improve cell viability and improve tissue regeneration by applying red light and/or NIR light stimulation with optional heating to specific region or majority of the body's skin surface. Red light/near infrared light (NTR) can activate mitochondrial function, increase ATP production and have deep tissue penetration (several centimeters). They can improve cell energy metabolism and improve/restore cell vitality. They can promote tissue, nerve growth and repair in wound healing. Its effect of improving blood and lymphatic circulation will also provide beneficial health effects. In some embodiments, the device can have a blanket/quilt or sleep bag or full body clothes type shape/structure/size that can cover most part of the human body or partial body clothes that can cover the trunk of the body. The device comprises flexible fabric sheet or membrane sheet. The skin contact side of the device has an array of light emitting unit such as LED or light bulb or laser. The wavelength of the light can be between 550-1100 nm such as those having peak wavelength at 590 nm or 670 nm or 780 nm or 790 nm or 808 nm or 810 nm or 850 nm or 980 nm or 1064 nm or their combinations. In some embodiments it is selected from red light/NIR such as those with peak at 633, 670, 810, 850, 980 and 1064 nm. In some embodiments it has a peak wavelength at 808-820 nm. In some embodiments it has a peak wavelength at 633 nm or 670 nm or 810 nm or 850 nm. In some embodiments the light emitting unit is 670 nm LED or 810 nm LED or 850 nm LED or their combinations. In some embodiments the light intensity can be 0.1-mW/cm² measured at skin when it is placed on top of human body. For example, the device can have multiple light emitting units such as 10-100 spatially evenly distributed LEDs having 0.1-6 W power level each with total power of 5-200 W, and the light output of each light emitting unit (e.g. LED) can be 10-500 lumens each. The light intensity and power level need to be comfortable to the user. Too high light intensity and power level that can damage the skin/tissue being irradiated should be avoided. The device can have a control that can adjust the treatment time and intensity. The device can have a light reflecting layer (e.g. an aluminum coated layer) facing the light emitting unit and skin to prevent light leaking to outside and keep most of the light towards the skin. The device can also have a ventilation means such as an air inlet connected to a fan to introduce airflow to the covered body space to avoid overheat. It can be powered by battery or a low voltage converter (e.g. 5V-36V) connected to 110V-220V electric outlet. To achieve desired effect, the user can cover specific region or most of part of the body with the device to receive red light/NIR irradiation for 5 min to 1 hour 1-3 times a day, either naked or in light permeable cloth, similar to indoor tanning but using red light/NIR instead of UV. In some embodiments, instead being a full body covering device the device has a flexible structure of muffler/scarf or a rigid structure similar to the device in FIG. 12 to be wrapped around neck to provide red light/NIR irradiation to neck area especially to carotid artery and jugular vein area to achieve these effects. The irradiation can be continuous or intermittent (e.g. 2 min on and 1 min off). It can also be pulse light irradiation at the frequency of brain wave described previously. In some embodiments the irradiation can be 1 min-100 min 1-3 times a day for 1-10 weeks or until desired effect is achieved. The intensity of the light needs to be not too high that can cause damage to the skin of the irradiation area. The light stimulation can be in the frequency pattern described previously (e.g. brain waves) or non-pulsed (continuous). Additional means that can provide sound or vibration stimulation can also be incorporated within, such as speaker or vibrator or inflatable air bag that can provide stimulation at gamma wave (35˜45 Hz), alpha wave (7.5-14 Hz), theta wave (4-7.5 Hz), delta wave (0.1-4 Hz) or their combinations as previously described. In some embodiments, the speaker or vibrator or inflatable air bag provide sound or vibration or other mechanical stimulation at the frequency selected from between 0.1 Hz to 10 Hz. In some embodiments, the speaker or vibrator or inflatable air bag provide sound or vibration or other mechanical stimulation at the frequency selected from between 10 Hz to 50 Hz. In some embodiments, the speaker or vibrator or inflatable air bag provide sound or vibration or other mechanical stimulation at the frequency selected from between 20 Hz to 150 Hz. In some embodiments, the speaker or vibrator or inflatable air bag provide sound or vibration or other mechanical stimulation at the frequency selected from between 100 Hz to 200 Hz. In some embodiments, the speaker or vibrator or inflatable air bag provide sound or vibration or other mechanical stimulation at the frequency selected from between 200 Hz to 500 Hz. In some embodiments, the speaker or vibrator or inflatable air bag provide sound or vibration or other mechanical stimulation at the frequency selected from between 500 Hz to 600 Hz. In some embodiments, the speaker produces a sound of cat purr or its mimic. In some embodiments, it further comprises heating means such as those described previously to provide heat stimulation to the body with skin contact surface between 40˜60° C. In some embodiments, the device further comprises electrical pulse stimulation means such as those described previously to provide electric pulse stimulation to the body. In some embodiments, it further comprises electrical and/or magnetic field stimulation generating means provide electric/magnetic field stimulation to the body. The light or sound or vibration or heat or electric or electric/magnetic field stimulation can be applied to selected specific regions such as acupuncture points or their nearby area or area covering lymph nodes or the area covering specific organ or all the region that the fabric can covers such as the whole body or trunk of the body.

The device can have a control means such as built-in circuit with control button/pad that can adjust the treatment time and intensity as previous described. The device can have a communication module that can communicate with an external control (command) module to receive commands of the stimulation output (e.g. stimulation type, time, frequency, on/off, power level and pulse pattern) and produce stimulation accordingly. The external control module can be a remote or computer or a cell phone with dedicated application installed. The communication can utilize Wi-Fi or blue tooth or IR or radio signal. The device can have an on/off control to turn on or turn off the communication module. The device can have a built-in power supply such as battery or be connected to an electricity outlet.

In some embodiments, the device is in a format of wearable garment such as waistcoat vest, sleeveless upper-body shirt, skirt, short pant, shorts, short sleeve cloth and etc. such as those shown in FIG. 20 top. In some embodiments, it has hat/hood and/or tall collar shape like component so it can provide stimulations to neck and head region as shown in the figure. As shown in FIG. 20 bottom, an examplary device is a vest like wearble device, multiple LED lights are incorporated within to face the skin. If the fabric is red light/NIR transparent or does not significantly block the light, the LED can be placed on the outer surface of the fabric. If it blocks the light passing, the LED will need to be placed on the inner surface of the fabric. The vest can have multiple pockets to hold the electric components of the device such as the control unit and power source. In one example, the LEDs used in the vest of FIG. 20 are 650-670 nm red light LED of 0.2 w/each and nm NIR LED of 0.1 w/each. A volunteers uses it 0.5-1 hr daily for a month and reports lower body fat, better skin condition, better immunity, improved energy level and better sleep quality.

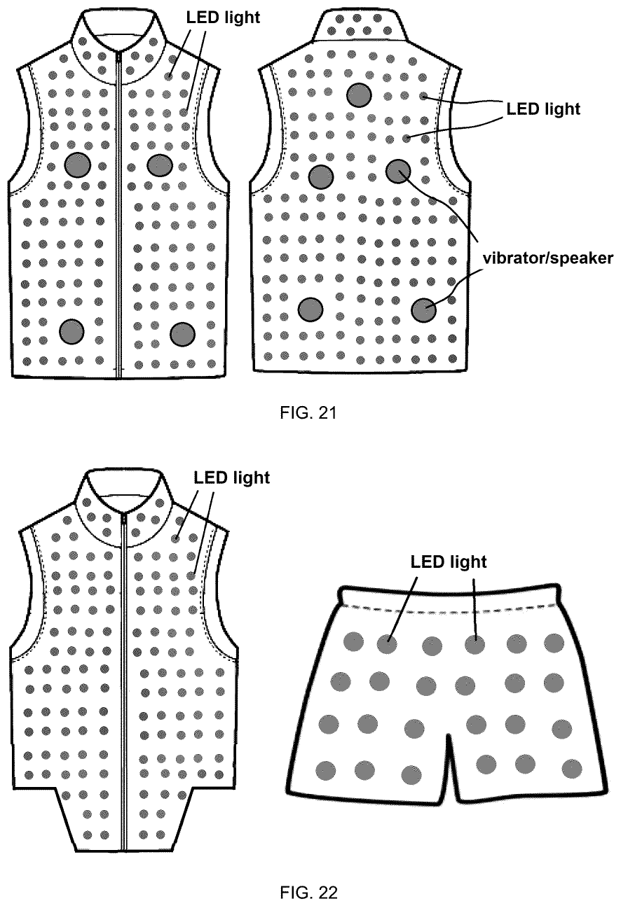

As shown in FIG. 21, one or more speakers or vibrators are placed on the vest to provide sound/vibration stimulations. In one example, the LEDs used in the vest of FIG. 21 are 650-nm red light LED of 0.2 w/each and 850 nm NIR LED of 0.1 w/each. The built in speakers produce gamma wave sound (40-50 Hz) or delta wave sound pulse modulated at 0.2-2 Hz. A volunteers uses it 0.5-1 hr daily for a month and reports lower body fat, better skin condition, better immunity, improved energy level, better sleep quality and better memory.

As shown in FIG. 22, additional extension at the bottom of the vest is added, which will provide stimulations to lower abdomen, lower back, anus area (to treat hemorrhoid and other digestive disorder such as constipation), pubic area and genital region. A device in a format of shorts as shown in FIG. 22 right can also be adopted to provide stimulation to these areas. It can further be used to improve sex hormone production, which can improve muscle growth and provide anti-aging effect. It can also improve gynecologic health for women. Instead of the format of shorts, it can also be in a shape of loincloth, or menstrual belt or diaper/diaper pant or panty liner format, which can provide light stimulation with LED attached on it and optional heat stimulation and other stimulations such as sound/vibrational stimulation to anus area, pubic area and genital region. It can be used relieve discomfort or improve health of these region or improve health systematically.

The current invention also disclose method to improve immunity by apply said red light/NIR stimulations with optional heating to the skin areas selected from those covering major lymph nodes, adenoid, tonsil, thymus and spleen. Additional above said sound/vibration and heat stimulations can also be applied. This will boost immune cells' activity, help to fight against aging, cancer and infections. The major lymph nodes can be selected from facial lymph nodes, cervical lymph nodes, supraclavicular lymph nodes, infraclavicular lymph nodes, epitrochlear lymph nodes, axillary lymph nodes (armpit lymph node), inguinal lymph nodes or their combinations. The device can be either a red light/NIR lamp or a device can be attached to the neck such as those described previously (e.g. similar to a scarf or those in FIG. 12) or a cloth like device as those described above with the stimulation means positioned to the above target areas or other format that can cover the lymph nodes. It can be a small pouch or soft pillow like device with optional fixing means such as band/strip suitable to be placed under armpit and placed on top of the inguinal lymph node area with the stimulations means incorporated within. It can be used by a cancer patient to treat cancer by increasing their immunity. The light irradiation and heating can be applied to tumor drain lymph node area or all major lymph nodes over the whole body. The user can use it to stimulate the target areas for 10 min to 2 hr daily. The light wavelength can be 550-1100 nm as previously described such as those having peak wavelength at 590 nm or 670 nm or nm or 790 nm or 808 nm or 810 nm or 850 nm or 980 nm or 1064 nm or their combinations. In some embodiments it is selected from red light/NIR (near IR) such as those with peak at 633, 670, 810, 850, 980 and 1064 nm. In some embodiments it has a peak wavelength at 808-820 nm. In some embodiments it has a peak wavelength at 633 nm or 670 nm or 810 nm or 850 nm. In some embodiments the light intensity can be 10-1000 mW/cm².

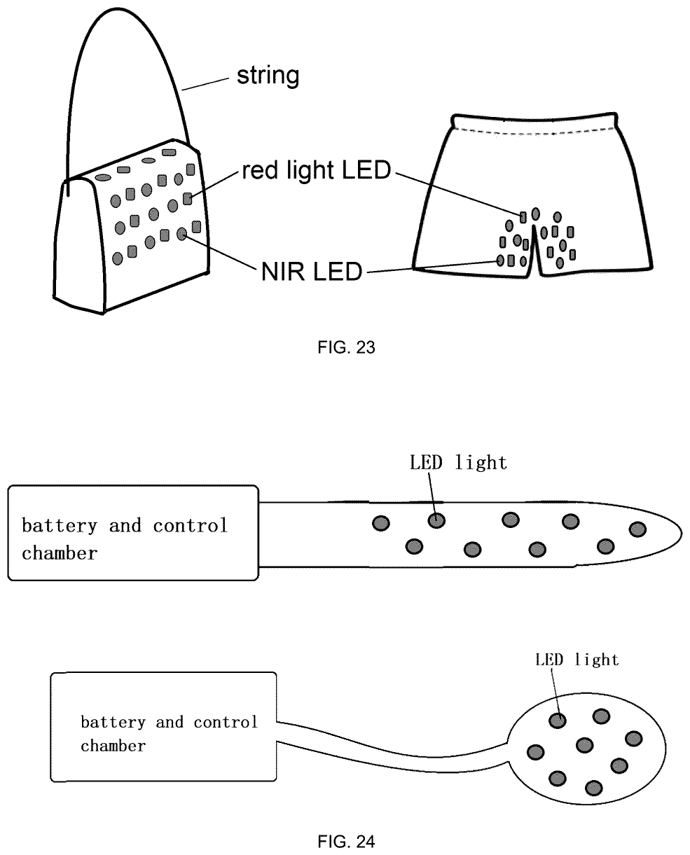

FIG. 23 left shows an example of the device in a small pillow shape to be placed under armpit to stimulate armpit lymph nodes. It has multiple 810 nm IR LED and 655 nm red light LED on its surface and built-in heater inside to maintain its surface temperature at 40˜55 degree. It also has an elastic string so the user can hang the string on shoulder to keep device under the arm. Speaker/vibrator can also be incorporated within to provide mechanical stimulation, which can be either at the brain wavelength disclosed previously or at higher frequency (e.g. 100 Hz˜10k Hz). FIG. 23 right shows an example of the device in the form of underwear, which has multiple NIR LED and red light LED on its skin contacting surface and a heating film to provide light and heat stimulation to a user's inguinal lymph node area. In one example a volunteer receives red light and NIR radiation to his armpit lymph node and inguinal lymph node area from 650-670 nm red light LEDs and 850 nm NIR LEDs at 10 w total power level, as well as heating stimulation to these area by maintaining skin contact surface between 40-50° C., 30 min-1 hr 1-3 times daily for a month. The volunteer reports improved immunity and energy level.

The said red light/NIR stimulations means (e.g. LED) described above can also be incorporated within a insertable device to be inserted in to mouth or ear or nose or vaginal or anus to apply said red light/NIR stimulations described above to the area inside those body chamber to treat infection, inflammation, tissue damage, ulcer and promote cell growth/tissue repair and nerve growth there. Said sound/vibration/heat stimulation producing means described previously can also be incorporated to apply said sound/vibration/heat stimulations described previously to these areas to improve the treatment efficacy. The device has one or more light transparent windows made of light transparent material to allow the light generated by LED inside to pass. Its structure can contain a bendable/flexible region to allow it to reach the area of interest, for example the area of infection or the lymph node area close to the infection site. As shown in FIG. 24 top, it contains a chamber having battery and control circuit inside and this chamber allow the user to hold it with hand. The insertable rod/tip part has LEDs inside to produce red light and/or NIR light stimulation with optional heating and vibration means inside. To be used for different body cavity, it can have different size, e.g. smaller size for ear and bigger size for mouse. It can be inserted to mouse to treat mouse ulcer by irradiating the ulcer region, and treat infection and inflammation by irradiating tonsil, or treat other oral infection such as gum/tooth infection by irradiating the infection area or the lymph node close to the infection site. It can be inserted into the ear to treat ear infection and improve hearing. It can also contains heating means to provide heat stimulation as well as means to provide mechanical stimulation such as sound or vibrational stimulation. In some embodiments the irradiation can be 5 min-30 min 1-3 times a day until desired effect is achieved. It can also be inserted into vaginal cavity to treat infection and inflammation there and improve reproductive system health. The lollipop shaped insertable device in FIG. 24 bottom has a flexible region to connect the light emitting part and the battery/control part so the user can bend it to reach the desired stimulation region. In some embodiments, the device contains multiple 650-670 nm red light LEDs and 810-850 nm NIR LEDs that can provide light radiation at 5-100 mW/cm² intensity and built-in heater to maintaining its skin contact surface between 40-50° C. to provide heat stimulation to the area treated. The stimulation pattern, time, temperature, light intensity and wavelength can be adjusted by a control means of the device. The device can also contain means to generate cooling stimulation (e.g. 5-15° C. to treated area) which can be applied to the treated area. The cooling stimulation can reduce pain and inflammation.

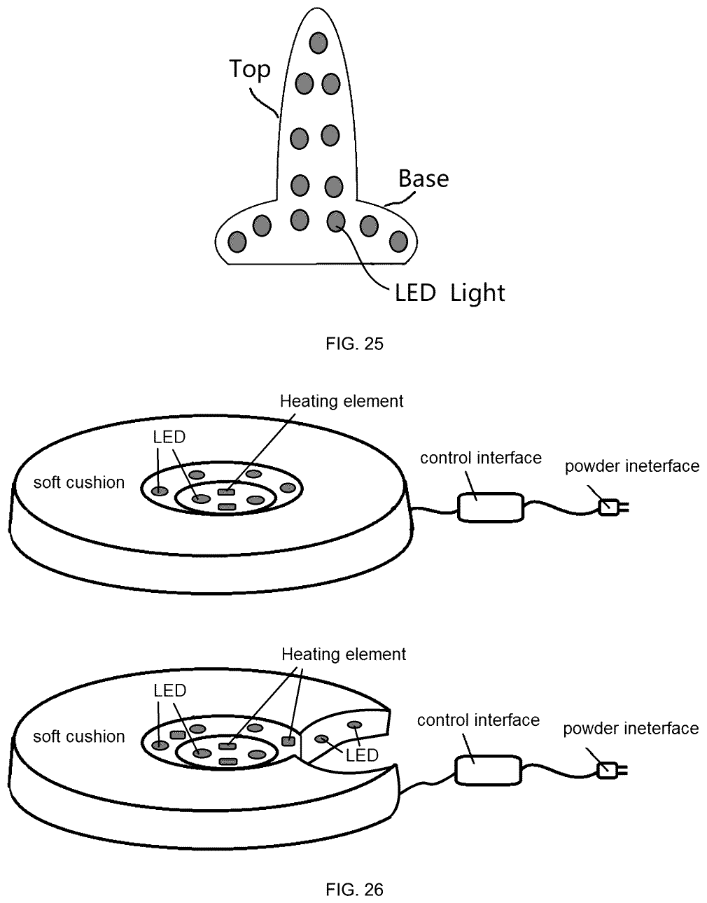

The device in FIG. 25 is to be inserted into anus to treat hemorrhoid. The top insertable part is to stimulate the inside of rectum to treat internal hemorrhoid and the base is to stimulate the outside area to treat external hemorrhoid. The device contains multiple red light LEDs and NIR LEDs that can provide light radiation at 5-100 mW/cm² intensity in some embodiments. It can further contain heating means to provide heat stimulation such as 38-50° C. heating to anus area as well as means to provide mechanical stimulation such as sound or vibrational stimulation. It can also be inserted to virginal to improve tissue repairing/healing and treat virginal disorder such as local infection and relieve discomfort. The stimulation pattern, time, temperature, light intensity and wavelength can be adjusted by a control means of the device. Another format of the device has a hemorrhoid seat cushion like configuration, to provide light and heat stimulation to anus area or genital region or both with optional vibrational stimulation to the skin contact area of butt when people sit on it. It has a soft cushion raised area to allow people to sit on it with optional vibrator and heating element inside to provide vibration and/or heating stimulation to the butt. The cushion can be any shape suitable for people to sit on it such as square or donut pillow shape. It also has a sunken concave area or a hole in the middle of the cushion to host one or more red light/NIR LED (e.g. at 600-900 nm wavelength) and heating elements such as heating lamp (e.g. FIR lamp), polymer PTC heating elements or ceramic PTC heater or those heating element previously described. It can further contain a fan to blow the hot air generated by the heating element to anus/genital region and prevent the device from overheating. When people sit on it, his anus/genital region will be on top and exposed to the concave/hole area to receive light stimulation and heat stimulation. The heat level should be the level not causing damage to the skin (e.g. temperature within 40˜60 degree) and can be controlled by a control interface. It can be used to improve tissue repairing/healing in the genital area and treat hemorrhoid or virginal disorder such as local infection and relieve discomforts, by e.g. 10-30 min a day at the stimulation level comfortable to the user. It can also be used to improve sex hormone production by irradiating genital area such as testicle. As shown in FIG. 26 top, an O ring shape soft seat cushion has a concave area in the middle. The concave area has multiple red light and/or NIR LED (e.g. of 600-nm as those used in other devices described previously) on the wall and the bottom as well as multiple heating elements on the bottom to host the anus/genital part of a user sitting on the cushion to provide light and heat stimulation to his anus/genital area. A control unit is attached to the device to control the stimulation pattern, intensity and time. The device can have one or more gap/opening on the cushion to allow air to circulate and vent hot air as well as to hold user's body better and provide more stimulation area to genital region as shown in FIG. 26 bottom. User can put it on the chair while working in the office to improve blood circulation, cell viability and promote tissue repair to relieve hemorrhoid pain and speed up healing. In one example a volunteer receives red light and NIR radiation from 650-670 nm red light LEDs and 850 nm NIR LEDs at 10 w total power level to anus/genital area as well as heating stimulation by maintaining device surface temperature between 40-50° C., 30 min-1 hr 1-3 times daily for a month. Improved healing at treated area and improved libido level are observed.

The devices described in the current invention can use heating stimulation. Alternatively, cooling stimulation or both heating and cooling stimulation can be employed sequentially such as rotating heating and cooling stimulation. The cooling effect can be achieved by using a built-in thermoelectric cooler such as Peltier element that can cool the skin contact surface at 5-15° C. or providing cold air flow to the target area. When the treated area has inflammation and pain, cooling stimulation can be applied.

In the current application, the “/” mark means “and” and/or “or” and/or their combination. Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known mechanics, instruments, methods and skills. A skilled person can easily find the knowledge from textbooks such as the textbooks, scientific journal papers and other well-known reference sources.

1. A method for improving memory and sleep quality in a subject in need thereof, the method comprising administering a non-invasive electrical stimulus having a frequency of about 35 Hz to about 45 Hz to the subject's skin surface when a subject is awake and a non-invasive electrical stimulus having a frequency of about 0.1 Hz to about 4 Hz to the subject's skin when a subject is in sleep.

2. The method of claim 1, wherein additional heat stimulation is applied to the head.

3. The method of claim 1, wherein additional infra-red light stimulation is applied to the head.

4. The method of claim 1, wherein additional mechanical stimulation having a frequency of about 35 Hz to about 45 Hz is applied when a subject is awake and mechanical stimulation having a frequency of about 0.1 Hz to about 4 Hz is applied when a subject is in sleep.

5. A device for improving memory and sleep quality in a subject in need, said device comprising:
one or more infra-red light emitting units facing the skin of the head;
one or more electrode assembly that can be attached to the skin of the head;
one or more speaker unit; and
a helmet shape housing,
wherein said electrode assembly and said speaker unit are connected to the helmet shape housing with electrical wire and said infra-red light emitting units are mounted on the skin facing surface of the helmet shape housing.

6. The device of claim 5, wherein the electrode assembly can generate an electric pulse at about 35 Hz to about 45 Hz or about 0.1 Hz to about 4 Hz.

7. The device of claim 5, wherein the speaker unit can generate a sound pulse at about 35 Hz to about 45 Hz or about 0.1 Hz to about 4 Hz.
